(19) United States
(12) Patent Application Publication
(10) Pub. No.: US 2021/0154359 A1
(43) Pub. Date: May 27, 2021

(54) FIBRILLAR-PROTEIN BASED COMPOSITIONS AND USES THEREOF

(71) Applicant: Hi-Aspect Limited, Lower Hutt (NZ)

(72) Inventors: Leonardo Negrón Ruíz, Lower Hutt (NZ); Manmeet Kaur, Lower Hutt (NZ); Laura Joy Domigan, Lower Hutt (NZ); Juliet Ann Gerrard, Lower Hutt (NZ)

(21) Appl. No.: 16/629,897

(22) PCT Filed: Jul. 14, 2017
(86) PCT No.: PCT/NZ2017/050094

§ 371 Date: Jan. 9, 2020

(51) Int. Cl. A61L 26/00; A61K 31/12; A61K 31/203; A61K 31/49; A61K 31/522; A61K 33/30; A61K 8/64; A61Q 17/04; A61Q 19/00

(52) U.S. Cl. CPC A61L 26/0066; A61K 31/12; A61K 31/203; A61K 31/49; A61Q 19/00; A61K 33/30; A61K 8/64; A61L 26/0047; A61Q 17/04; A61K 31/522

(57) ABSTRACT

The present invention relates to a composition comprising a protein fibril scaffold, wherein the composition permits the attachment and extended release of agents such as biologically active agents, and thus has numerous applications, including in skin care and in wound care.

### TECHNICAL FIELD

The present invention relates to proteinaceous compositions that permit the attachment and extended release of agents, and thus have numerous applications, including in skin care and in wound care.

### BACKGROUND OF THE INVENTION

There exists a need in the market for products that can receive and subsequently release agents in an extended manner, including biologically active agents. Where those products are formulated for topical administration to keratinaceous surfaces (e.g. skin, hair, nails) or to wounds, then it can also be important to ensure that the products are suitable for such use in that they possess the right adhesion properties so that they adhere to the surface appropriately, and moreover meet the necessary safety requirements for use on the desired surface.

The present invention provides proteinaceous compositions suitable for topical application to keratinaceous surfaces (e.g. skin, hair, nails) and to wounds, wherein the proteinaceous compositions are capable of receipt and extended release of agents, and/or to at least provide the public with a useful choice.

### SUMMARY OF THE INVENTION

The inventions described and claimed herein have many attributes and embodiments including, but not limited to, those set forth or described or referenced in this Summary of the Invention. It is not intended to be all-inclusive and the inventions described and claimed herein are not limited to or by the features or examples identified in this Summary of the Invention, which is included for purposes of illustration only and not restriction.

In one aspect of the present invention there is provided a composition comprising a proteinaceous component, wherein the proteinaceous component comprises fibrillar protein, wherein the fibrillar protein comprises a scaffold. Suitably, the composition provides for attachment of an agent. More suitably, the composition provides for extended release of the agent.

In another aspect of the present invention, there is provided a composition comprising a proteinaceous component and an agent, wherein the proteinaceous component comprises fibrillar protein, wherein the fibrillar protein comprises a scaffold. Suitably, the composition provides for extended release of the agent.

The compositions of the present invention may comprise an oil in water emulsion. The compositions of the present invention may comprise an oil in water in oil emulsion.

The compositions of the present invention may be formulated for administration to a keratinaceous surface. Suitably, the keratinaceous surface comprises skin, nail, or hair.

In yet another aspect, the present invention provides a skincare composition comprising the composition of the present invention.

In yet another aspect, the present invention provides a nail care composition comprising the composition of the present invention.

In yet another aspect, the present invention provides a hair care composition comprising the composition of the present invention.

In yet another aspect, the present invention provides a sunscreen composition comprising the composition of the present invention.

In yet another aspect, the present invention provides a wound care composition comprising the composition of the present invention.

In another aspect of the present invention, there is provided the use of a fibrillar protein scaffold in the manufacture of a skincare composition. In yet another aspect of the present invention, there is provided the use of a fibrillar protein scaffold in the manufacture of a composition for use in skin care.

Yet another aspect of the present invention provides a method of caring for a keratinaceous surface in a subject, wherein the method comprises administering a composition of the present invention to the keratinaceous surface of the subject. Suitably, the composition comprises a fibrillar protein scaffold and an agent. In some embodiments, the keratinaceous surface comprises skin, hair, and/or nail.

In another aspect of the present invention, there is provided the use of a fibrillar protein scaffold in the manufacture of a suncare composition. In yet another aspect of the present invention, there is provided the use of a fibrillar protein scaffold in the manufacture of a composition for use in sun care.

Yet another aspect of the present invention provides a method of protecting a keratinaceous surface in a subject from UV radiation, wherein the method comprises administering a composition of the present invention to the keratinaceous surface of the subject. Suitably, the composition comprises a fibrillar protein scaffold and an agent. Suitable keratinaceous surfaces include skin, hair, and/or nail.

In another aspect of the present invention, there is provided the use of a fibrillar protein scaffold in the manufacture of a wound care composition. In yet another aspect of the present invention, there is provided the use of a fibrillar protein scaffold in the manufacture of a composition for use in wound care.

Yet another aspect of the present invention provides a method of protecting or treating a wound in a subject, wherein the method comprises administering a composition of the present invention to the wound of the subject. Suitably, the composition comprises a fibrillar protein scaffold and an agent.

Accordingly, as a wound care composition, the composition of the present invention may comprise an agent for preventing and/or treating an infection in a wound, and/or for preventing and/or reducing inflammation in a wound, and/or for promoting or stimulating wound healing.

Another aspect of the present invention thus provides a method of preventing and/or treating an infection in a wound, and/or for preventing and/or reducing inflammation in a wound, and/or for promoting or stimulating wound healing, wherein the method comprises administering a composition of the present invention to the wound of the subject. Suitably, the composition comprises a fibrillar protein scaffold and an agent.

Another aspect of the present invention provides a skincare composition, a sun care composition or a wound care composition, wherein the composition comprises a proteinaceous component and an agent, wherein the proteinaceous component comprises 5-50 wt % fibrillar protein, and wherein the fibrillar protein comprises a scaffold. Suitably, the proteinaceous component comprises 10-40 wt % fibrillar protein. Suitably, the fibrillar protein is derived from whey protein and/or soy protein. More suitably, the fibrillar protein is derived from beta-lactoglobulin. Suitably, the composition comprises a shear thinning gel. More suitably, the composition comprises a thixotropic gel. Suitable agents include, but are not limited to a moisturising agent, a hydrating agent, a lipid, an oil, a vitamin, a mineral, a plant extract, a herbal extract, an anti-acne agent, an agent providing a health benefit, an agent which blocks, reduces or minimises the absorbance of ultraviolet radiation by the skin (including but not limited to a zinc-containing agent, a titanium-containing agent, a suitable plant extract, avobenzone), an agent for preventing and/or treating an infection, an agent for preventing and/or reducing inflammation, an agent for promoting or stimulating wound healing, an antibiotic, an antimicrobial, an agent suitable for the formulation of a skincare composition (including, but not limited to, a buffer, an acid, a base, vegetable glycerin, an emulsifier, a thickener, a preservative, a lubricant, a pigment, and/or a fragrance). Exemplary agents include, but are not limited to, vitamin A, retinol, retinyl palmitate, retinaldehyde, vitamin C, ascorbic acid, vitamin E, tocopherol, alpha tocopherol, hyaluronic acid, hyaluronan, sodium hyaluronate, collagen, keratin, plant oil, vegetable oil, fruit oil, olive oil, avocado oil, canola oil, grapeseed oil, jojoba oil, almond oil, sweet almond oil, hazelnut oil, sunflower seed oil, coconut oil, argan oil, emu oil, tamanu oil, neem oil, apricot kernel oil, rose hip seed oil, evening primrose oil, castor oil, glycerin, vegetable glycerin, bee venom, a bee venom derivative, honey, Manuka honey, a beta-hydroxy acid, salicylic acid, capric/caprylic triglyceride, caffeine, alpha-hydroxy acid, glycolic acid, lactic acid, fruit acid, cetyl alcohol, stearyl alcohol, and/or cetearyl alcohol, zinc oxide, titanium oxide, avobenzone, an extract of Gingko biloba, silymarin, an extract of pomegranate, an extract of Polypodium leucotomos, an extract of peach, an extract of moldavian dragonhead, an extract of Viola tricolor, an extract of carrot, carrot seed oil, wheat germ oil, an extract of Symplocos racemosa, aloe vera, an extract of aloe vera, an extract of basil, tumeric, an essential oil, propolis, curcumin, butyl methoxydibenzoylmethane, sodium hydroxide, lactic acid, silver, an antimicrobial peptide, a matrix protein, collagen, a growth factor, an alginate, a hydrocolloid, a hydrogel, and/or a nonsteroidal anti-inflammatory drug (NSAID). In suitable embodiments, the agent is selected from the group consisting of vitamin A, vitamin C, vitamin E, hyaluronic acid, sodium hyaluronate, a plant oil, glycerin, salicylic acid, capric/caprylic triglyceride, caffeine, alpha-hydroxy acid, glycolic acid, lactic acid, cetyl alcohol, zinc oxide, avobenzone, an antibiotic, an antimicrobial, a growth factor, a hydrocolloid, and a nonsteroidal anti-inflammatory drug (NSAID).

Yet another aspect of the present invention provides a composition of the present invention for use in in skincare, sun care, and/or wound care.

Yet another aspect of the present invention provides the use of a composition comprising a proteinaceous component and an agent in the manufacture of a skincare composition, a sun care composition or a wound care composition, wherein the proteinaceous component comprises 5-50 wt % fibrillar protein, and wherein the fibrillar protein comprises a scaffold. Suitably, the proteinaceous component comprises 10-40 wt % fibrillar protein. Suitably, the fibrillar protein is derived from whey protein and/or soy protein. More suitably, the fibrillar protein is derived from beta-lactoglobulin. Suitably, the composition comprises a shear thinning gel. More suitably, the composition comprises a thixotropic gel. Suitable agents include, but are not limited to a moisturising agent, a hydrating agent, a lipid, an oil, a vitamin, a mineral, a plant extract, a herbal extract, an anti-acne agent, an agent providing a health benefit, an agent which blocks, reduces or minimises the absorbance of ultraviolet radiation by the skin (including but not limited to a zinc-containing agent, a titanium-containing agent, a suitable plant extract, avobenzone), an agent for preventing and/or treating an infection, an agent for preventing and/or reducing inflammation, an agent for promoting or stimulating wound healing, an antibiotic, an antimicrobial, an agent suitable for the formulation of a skincare composition (including, but not limited to, a buffer, an acid, a base, vegetable glycerin, an emulsifier, a thickener, a preservative, a lubricant, a pigment, and/or a fragrance). Exemplary agents include, but are not limited to, vitamin A, retinol, retinyl palmitate, retinaldehyde, vitamin C, ascorbic acid, vitamin E, tocopherol, alpha tocopherol, hyaluronic acid, hyaluronan, sodium hyaluronate, collagen, keratin, plant oil, vegetable oil, fruit oil, olive oil, avocado oil, canola oil, grapeseed oil, jojoba oil, almond oil, sweet almond oil, hazelnut oil, sunflower seed oil, coconut oil, argan oil, emu oil, tamanu oil, neem oil, apricot kernel oil, rose hip seed oil, evening primrose oil, castor oil, glycerin, vegetable glycerin, bee venom, a bee venom derivative, honey, Manuka honey, a beta-hydroxy acid, salicylic acid, capric/caprylic triglyceride, caffeine, alpha-hydroxy acid, glycolic acid, lactic acid, fruit acid, cetyl alcohol, stearyl alcohol, and/or cetearyl alcohol, zinc oxide, titanium oxide, avobenzone, an extract of Gingko biloba, silymarin, an extract of pomegranate, an extract of Polypodium leucotomos, an extract of peach, an extract of moldavian dragonhead, an extract of Viola tricolor, an extract of carrot, carrot seed oil, wheat germ oil, an extract of Symplocos racemosa, aloe vera, an extract of aloe vera, an extract of basil, tumeric, an essential oil, propolis, curcumin, butyl methoxydibenzoylmethane, sodium hydroxide, lactic acid, silver, an antimicrobial peptide, a matrix protein, collagen, a growth factor, an alginate, a hydrocolloid, a hydrogel, and/or a nonsteroidal anti-inflammatory drug (NSAID). In suitable embodiments, the agent is selected from the group consisting of vitamin A, vitamin C, vitamin E, hyaluronic acid, sodium hyaluronate, a plant oil, glycerin, salicylic acid, capric/caprylic triglyceride, caffeine, alpha-hydroxy acid, glycolic acid, lactic acid, cetyl alcohol, zinc oxide, avobenzone, an antibiotic, an antimicrobial, a growth factor, a hydrocolloid, and a nonsteroidal anti-inflammatory drug (NSAID).

Yet another aspect of the present invention provides the use of a composition comprising a proteinaceous component and an agent in the manufacture of a composition for caring for a keratinaceous surface, for protecting a keratinaceous surface from UV radiation, for protecting a wound, for treating a wound, for preventing an infection in a wound, for treating an infection in a wound, for preventing inflammation in a wound, for reducing inflammation in a wound, for promoting wound healing, and/or for stimulating wound healing, wherein the proteinaceous component comprises 5-50 wt % fibrillar protein, and wherein the fibrillar protein comprises a scaffold. Suitably, the proteinaceous component comprises 10-40 wt % fibrillar protein. Suitably, the fibrillar protein is derived from whey protein and/or soy protein. More suitably, the fibrillar protein is derived from beta-lactoglobulin. Suitably, the composition comprises a shear thinning gel. More suitably, the composition comprises a thixotropic gel. Suitable agents include, but are not limited to a moisturising agent, a hydrating agent, a lipid, an oil, a vitamin, a mineral, a plant extract, a herbal extract, an anti-acne agent, an agent providing a health benefit, an agent which blocks, reduces or minimises the absorbance of ultraviolet radiation by the skin (including but not limited to a zinc-containing agent, a titanium-containing agent, a suitable plant extract, avobenzone), an agent for preventing and/or treating an infection, an agent for preventing and/or reducing inflammation, an agent for promoting or stimulating wound healing, an antibiotic, an antimicrobial, an agent suitable for the formulation of a skincare composition (including, but not limited to, a buffer, an acid, a base, vegetable glycerin, an emulsifier, a thickener, a preservative, a lubricant, a pigment, and/or a fragrance). Exemplary agents include, but are not limited to, vitamin A, retinol, retinyl palmitate, retinaldehyde, vitamin C, ascorbic acid, vitamin E, tocopherol, alpha tocopherol, hyaluronic acid, hyaluronan, sodium hyaluronate, collagen, keratin, plant oil, vegetable oil, fruit oil, olive oil, avocado oil, canola oil, grapeseed oil, jojoba oil, almond oil, sweet almond oil, hazelnut oil, sunflower seed oil, coconut oil, argan oil, emu oil, tamanu oil, neem oil, apricot kernel oil, rose hip seed oil, evening primrose oil, castor oil, glycerin, vegetable glycerin, bee venom, a bee venom derivative, honey, Manuka honey, a beta-hydroxy acid, salicylic acid, capric/caprylic triglyceride, caffeine, alpha-hydroxy acid, glycolic acid, lactic acid, fruit acid, cetyl alcohol, stearyl alcohol, and/or cetearyl alcohol, zinc oxide, titanium oxide, avobenzone, an extract of Gingko biloba, silymarin, an extract of pomegranate, an extract of Polypodium leucotomos, an extract of peach, an extract of moldavian dragonhead, an extract of Viola tricolor, an extract of carrot, carrot seed oil, wheat germ oil, an extract of Symplocos racemosa, aloe vera, an extract of aloe vera, an extract of basil, tumeric, an essential oil, propolis, curcumin, butyl methoxydibenzoylmethane, sodium hydroxide, lactic acid, silver, an antimicrobial peptide, a matrix protein, collagen, a growth factor, an alginate, a hydrocolloid, a hydrogel, and/or a nonsteroidal anti-inflammatory drug (NSAID). In suitable embodiments, the agent is selected from the group consisting of vitamin A, vitamin C, vitamin E, hyaluronic acid, sodium hyaluronate, a plant oil, glycerin, salicylic acid, capric/caprylic triglyceride, caffeine, alpha-hydroxy acid, glycolic acid, lactic acid, cetyl alcohol, zinc oxide, avobenzone, an antibiotic, an antimicrobial, a growth factor, a hydrocolloid, and a nonsteroidal anti-inflammatory drug (NSAID).

Yet another aspect of the present invention provides a method for caring for a keratinaceous surface and/or for protecting a keratinaceous surface of a subject from UV radiation, wherein the method comprises administering a composition to the keratinaceous surface of the subject, wherein the composition comprises a proteinaceous component and an agent, wherein the proteinaceous component comprises 5-50 wt % fibrillar protein, and wherein the fibrillar protein comprises a scaffold. Suitably, the proteinaceous component comprises 10-40 wt % fibrillar protein. Suitably, the fibrillar protein is derived from whey protein and/or soy protein. More suitably, the fibrillar protein is derived from beta-lactoglobulin. Suitably, the composition comprises a shear thinning gel. More suitably, the composition comprises a thixotropic gel. Suitable agents include, but are not limited to a moisturising agent, a hydrating agent, a lipid, an oil, a vitamin, a mineral, a plant extract, a herbal extract, an anti-acne agent, an agent providing a health benefit, an agent which blocks, reduces or minimises the absorbance of ultraviolet radiation by the skin (including but not limited to a zinc-containing agent, a titanium-containing agent, a suitable plant extract, avobenzone), an agent for preventing and/or treating an infection, an agent for preventing and/or reducing inflammation, an agent for promoting or stimulating wound healing, an antibiotic, an antimicrobial, an agent suitable for the formulation of a skincare composition (including, but not limited to, a buffer, an acid, a base, vegetable glycerin, an emulsifier, a thickener, a preservative, a lubricant, a pigment, and/or a fragrance). Exemplary agents include, but are not limited to, vitamin A, retinol, retinyl palmitate, retinaldehyde, vitamin C, ascorbic acid, vitamin E, tocopherol, alpha tocopherol, hyaluronic acid, hyaluronan, sodium hyaluronate, collagen, keratin, plant oil, vegetable oil, fruit oil, olive oil, avocado oil, canola oil, grapeseed oil, jojoba oil, almond oil, sweet almond oil, hazelnut oil, sunflower seed oil, coconut oil, argan oil, emu oil, tamanu oil, neem oil, apricot kernel oil, rose hip seed oil, evening primrose oil, castor oil, glycerin, vegetable glycerin, bee venom, a bee venom derivative, honey, Manuka honey, a beta-hydroxy acid, salicylic acid, capric/caprylic triglyceride, caffeine, alpha-hydroxy acid, glycolic acid, lactic acid, fruit acid, cetyl alcohol, stearyl alcohol, and/or cetearyl alcohol, zinc oxide, titanium oxide, avobenzone, an extract of Gingko biloba, silymarin, an extract of pomegranate, an extract of Polypodium leucotomos, an extract of peach, an extract of moldavian dragonhead, an extract of Viola tricolor, an extract of carrot, carrot seed oil, wheat germ oil, an extract of Symplocos Racemosa, Aloe Vera, an Extract of Aloe Vera, an Extract of Basil, Tumeric, an essential oil, propolis, curcumin, butyl methoxydibenzoylmethane, sodium hydroxide, lactic acid, silver, an antimicrobial peptide, a matrix protein, collagen, a growth factor, an alginate, a hydrocolloid, a hydrogel, and/or a nonsteroidal anti-inflammatory drug (NSAID). In suitable embodiments, the agent is selected from the group consisting of vitamin A, vitamin C, vitamin E, hyaluronic acid, sodium hyaluronate, a plant oil, glycerin, salicylic acid, capric/caprylic triglyceride, caffeine, alpha-hydroxy acid, glycolic acid, lactic acid, cetyl alcohol, zinc oxide, avobenzone, an antibiotic, an antimicrobial, a growth factor, a hydrocolloid, and a nonsteroidal anti-inflammatory drug (NSAID).

Yet another aspect of the present invention provides a method for protecting a wound, for treating a wound, for preventing an infection in a wound, for treating an infection in a wound, for preventing inflammation in a wound, for reducing inflammation in a wound, for promoting wound healing, and/or for stimulating wound healing in a subject, wherein the method comprises administering a composition to the wound of the subject, wherein the composition comprises a proteinaceous component and an agent, wherein the proteinaceous component comprises 5-50 wt % fibrillar protein, and wherein the fibrillar protein comprises a scaffold. Suitably, the proteinaceous component comprises 10-40 wt % fibrillar protein. Suitably, the fibrillar protein is derived from whey protein and/or soy protein. More suitably, the fibrillar protein is derived from beta-lactoglobulin. Suitably, the composition comprises a shear thinning gel. More suitably, the composition comprises a thixotropic gel. Suitable agents include, but are not limited to a moisturising agent, a hydrating agent, a lipid, an oil, a vitamin, a mineral, a plant extract, a herbal extract, an anti-acne agent, an agent providing a health benefit, an agent which blocks, reduces or minimises the absorbance of ultraviolet radiation by the skin (including but not limited to a zinc-containing agent, a titanium-containing agent, a suitable plant extract, avobenzone), an agent for preventing and/or treating an infection, an agent for preventing and/or reducing inflammation, an agent for promoting or stimulating wound healing, an antibiotic, an antimicrobial, an agent suitable for the formulation of a skincare composition (including, but not limited to, a buffer, an acid, a base, vegetable glycerin, an emulsifier, a thickener, a preservative, a lubricant, a pigment, and/or a fragrance). Exemplary agents include, but are not limited to, vitamin A, retinol, retinyl palmitate, retinaldehyde, vitamin C, ascorbic acid, vitamin E, tocopherol, alpha tocopherol, hyaluronic acid, hyaluronan, sodium hyaluronate, collagen, keratin, plant oil, vegetable oil, fruit oil, olive oil, avocado oil, canola oil, grapeseed oil, jojoba oil, almond oil, sweet almond oil, hazelnut oil, sunflower seed oil, coconut oil, argan oil, emu oil, tamanu oil, neem oil, apricot kernel oil, rose hip seed oil, evening primrose oil, castor oil, glycerin, vegetable glycerin, bee venom, a bee venom derivative, honey, Manuka honey, a beta-hydroxy acid, salicylic acid, capric/caprylic triglyceride, caffeine, alpha-hydroxy acid, glycolic acid, lactic acid, fruit acid, cetyl alcohol, stearyl alcohol, and/or cetearyl alcohol, zinc oxide, titanium oxide, avobenzone, an extract of Gingko biloba, silymarin, an extract of pomegranate, an extract of Polypodium leucotomos, an extract of peach, an extract of moldavian dragonhead, an extract of Viola tricolor, an extract of carrot, carrot seed oil, wheat germ oil, an extract of Symplocos racemosa, aloe vera, an extract of aloe vera, an extract of basil, tumeric, an essential oil, propolis, curcumin, butyl methoxydibenzoylmethane, sodium hydroxide, lactic acid, silver, an antimicrobial peptide, a matrix protein, collagen, a growth factor, an alginate, a hydrocolloid, a hydrogel, and/or a nonsteroidal anti-inflammatory drug (NSAID). In suitable embodiments, the agent is selected from the group consisting of vitamin A, vitamin C, vitamin E, hyaluronic acid, sodium hyaluronate, a plant oil, glycerin, salicylic acid, capric/caprylic triglyceride, caffeine, alpha-hydroxy acid, glycolic acid, lactic acid, cetyl alcohol, zinc oxide, avobenzone, an antibiotic, an antimicrobial, a growth factor, a hydrocolloid, and a nonsteroidal anti-inflammatory drug (NSAID).

### BRIEF DESCRIPTION OF THE FIGURES

FIG. 1 provides transmission electron microscopy (TEM) images of two of the protein compositions prepared as described in Example 1, where A) is protein composition A and B) is protein composition C, wherein the protein fibrillar scaffold form is evident.

FIG. 2 shows the gel viscosities of protein composition B, protein composition C, and a Newtonian liquid, all of which were subjected to shear as described in Example 1. The viscosities were measured at 21° C. with Brookfield DV3T, spindle SC4-21, at 100 rpm. The dark triangles (▴) represent the Newtonian liquid, the squares ( [symbol] ) represent protein composition C, the grey circles ( [symbol] ) represent protein composition B. The x-axis refers to the time that the protein composition was in the viscometer.

FIG. 3 shows the effect of static shear and rest on the viscosity of protein composition C, as described in Example 1, where: the dark triangles (┐) represent the initial viscosity profile of the protein composition C; the grey squares ( [symbol] ) represent the viscosity profile of protein composition C after 1 minute rest; and the grey diamonds ( [symbol] ) represent the viscosity profile of protein composition C after 24-hours rest. Gel viscosities were measured at 21° C. with Brookfield DV3T, spindle SC4-21, at 200 rpm. The x-axis refers to the time that the protein composition was in the viscometer.

FIG. 4 shows the release profile of quinine sulphate in protein composition A and protein composition B compared to solutions of whey protein isolate (WPI) at equivalent protein concentrations of each, as described in Example 2, where the black bars represent protein composition A, the grey bars represent the WPI solution with an equivalent protein concentration (10 mg/mL), the bars with horizontal lines represent protein composition B, and the bars with diagonal lines represent the WPI solution with an equivalent protein concentration (40 mg/mL). Each bar represents the % of quinine sulphate released from the protein composition or WPI solution over time (% QS release refers to the percentage of quinine sulphate released over time). Each sample was measured in triplicate and the error bars represent the standard deviation of the mean.

FIG. 5 shows the release profile of quinine sulphate in protein composition C compared to quinine sulphate in a solution of whey protein isolate (WPI) at an equivalent protein concentration, as described in Example 2, where the grey (lighter) bars represent the % of quinine sulphate released from protein composition C and the black (darker) bars represent the % of quinine sulphate released from the WPI solution (% QS release refers to the percentage of quinine sulphate released over time). Each sample was measured in triplicate and the error bars represent the standard deviation of the mean.

FIG. 6 shows the release profile of quinine sulphate in protein composition C compared to quinine sulphate in commercially available gel sodium gluconate, as described in Example 2, where the grey (lighter) bars represent the % of quinine sulphate released from protein composition C and the black (darker) bars represent the % of quinine sulphate released from the commercially available gel (% QS release refers to the percentage of quinine sulphate released over time). Each sample was measured in triplicate and the error bars represent the standard deviation of the mean.

FIG. 7 shows the release profile of caffeine in protein composition C compared to a caffeine solution in PBS buffer (assay control) where the grey (lighter) bars represent the % of caffeine released from protein composition C and the black (darker) bars represent the % of caffeine released from the PBS buffer, as described in Example 2. Each sample was measured in triplicate and the error bars represent the standard deviation of the mean.

FIG. 8 shows a comparison of the fluorescence intensity (270 nm, 450 nm excitation and emission respectively) between the protein composition and a control (whey protein isolate—WPI) in the absence (white columns) and presence (patterned columns) of 100 mg/L zinc oxide nanoparticles (ZnO NP) dispersion, as described in Example 2. A decrease in the fluorescence intensity for both the protein composition (Δ431 RFU) and the WPI control (Δ210 RFU) upon exposure to the ZnO NPs was observed, indicating that both the protein composition and the WPI control can bind ZnO NPs.

FIG. 9 shows the absorbance spectra of the protein composition in the presence of ZnO NPs (dashed black line) and in the absence of ZnO NPs (dashed grey line) was recorded, as described in Example 2. The absorbance spectra of ZnO NPs is shown with the solid grey line.

FIG. 10 shows the diffusion rate of avobonezone from an emulsion in the presence and absence of the protein composition after 24 hours (white columns) and 72 hours (patterned columns), as described in Example 2, where it can be observed that an emulsion of avobenzone without the protein composition diffused 2.7 times faster than an emulsion of avobenzone with the protein composition.

FIG. 11 shows the release of avobenzone by protein composition C and an oil solution where the darker bars represent the % of avobenzone released from protein composition C and the lighter bars represent the % of avobenzone released from the oil solution.

FIG. 12 shows tissue viability results of the MTT assay as described in Example 3 where the mean tissue viability (% of negative control) is shown for the negative control (DPBS), the positive control (5% SDS), and protein composition C.

FIG. 13 shows the results of the skin adhesion study as described in Example 3, where the protein composition and whey protein isolate (WPI) were separately applied to a skin mimetic and subjected to wash cycles (both 1 wash and overnight). The solid line represents the amount of the protein composition and the dashed line represents the amount of the WPI retained on the skin mimetic after 1 wash (“After 1 wash”) and after overnight wash (“After o/n wash”).

FIG. 14 shows the light transmittance through skin mimetics after four compositions were spread over the mimetics, and the mimetics had then been subjected to washing, as described in Example 3. The four compositions comprised: commercially available sunscreen formulation X with 24.5% zinc oxide (labelled “Sunscreen X” and represented by a dashed grey line); commercially available sunscreen formulation Y with 15% zinc oxide (labelled “Sunscreen Y” and represented by a solid grey line); protein composition C made as per Example 1 with 15% zinc oxide (labelled “Protein comp C+ZnO” and represented by the dashed black line); and the protein composition containing soy protein manufactured using the method described in Example 1 with 15% zinc oxide and with 3% avobenzone (labelled “Soy protein comp+ZnO+avo” and represented by the solid black line).

FIG. 15 shows the percentage UVA blockage (or protection) of zinc oxide containing compositions remaining after several washes that was determined as described in Example 3, where FIG. 15A provides a comparison of a protein composition made using the method of Example 1 containing whey protein, 10% ZnO and 3% avobenzone (represented by the darker bars) and a commercially available sunscreen comprising an emulsion containing ZnO (commercially available sunscreen formulation Y with 15% zinc oxide, labelled “Sunscreen Y” and represented by the lighter bars); FIG. 15B provides a comparison of a protein composition made using the method of Example 1 containing whey protein, 10% ZnO and 3% avobenzone (represented by the darker bars), and another commercially available sunscreen comprising an emulsion containing ZnO (commercially available sunscreen formulation X with 24.5% zinc oxide, labelled “Sunscreen X” and represented by the lighter bars) and; and FIG. 15C provides a comparison of a protein composition made using the method of Example 1 containing soy protein fibrils, 10% ZnO and 3% avobenzone (represented by the darker bars), and the commercially available sunscreen formulation X with 24.5% zinc oxide (labelled “Sunscreen X” and represented by the lighter bars).

FIG. 16 shows the results of Example 5, where it can be seen that after 3 hours of incubation, the protein compositions selectively inhibited MMP2 and MMP9 without inhibiting MMP1. In FIG. 16, the % activity=(slope control/slope test sample)×100. In FIG. 16, three separate protein composition C were tested against each of MMP1, MMP2 and MMP9 and are represented by the circle, square and triangle icons, where the circle icon represents a sample of protein composition C that was produced on a small-scale (laboratory bench top) basis, and the square and triangle icons represent samples of protein composition C taken from separate scaled-up batches of protein composition C.

### GENERAL DEFINITIONS

Unless specifically defined otherwise, all technical and scientific terms used herein shall be taken to have the same meaning as commonly understood by one of ordinary skill in the art (for example, in protein chemistry, biochemistry, dermatology, immunology, and immunohistochemistry).

It is intended that reference to a range of numbers disclosed herein (e.g. 1 to 10) also incorporates reference to all related numbers within that range (e.g. 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

### DETAILED DESCRIPTION

### Compositions of the Invention

The present invention is based, at least in part, on the unexpected discovery that protein fibrils form into scaffolds that permit the attachment of a wide range of agents and subsequently provide the extended release of the agents. Without wishing to be bound by theory, it is believed that the large particle size, high surface area, and unique hydrophilic and lipophilic surface composition of the fibrillar scaffold provides it with these attachment and release properties. It is considered desirable to provide compositions comprising a fibrillar protein in scaffold form for many applications as described further herein.

Accordingly in one aspect of the present invention there is provided a composition comprising a proteinaceous component, wherein the proteinaceous component comprises a fibrillar protein, and wherein the fibrillar protein comprises a scaffold. In another aspect of the invention there is provided a composition comprising a proteinaceous component, wherein the proteinaceous component comprises a fibrillar protein in scaffold form.

The terms “fibrillar protein”, “protein fibril”, “protein nanofibril”, “fibril”, “fibrillar”, and the like can be used interchangeably and refer to proteinaceous material in fibril form. Protein fibrils are supramolecular, elongated structures formed by self-assembly from proteinaceous material which has lost the native conformation of the protein from which it is derived, such as denatured proteins and peptides (Nelson and Eisenberg, 2006; Hettiarachchi, C. A., 2013). As used herein, the term “scaffold” refers to an aggregate of protein fibrils, for instance an aggregate of protein fibrils which forms by self-assembly during or following the formation of protein fibrils.

Protein fibrils share characteristics, including structural similarity at the nanoscale (Sunde et al. 1997). Protein fibrils are generally non-water soluble, slow-degrading and partially resistant to proteolysis. One category of protein fibrils are amyloid fibrils, or amyloid-like fibrils, including fibrils from food-related proteins. Examples of amyloid or amyloid-like fibrils include those derived from:
milk proteins, such as beta-lactoglobulin (Arnaudov et al., 2003; Gosal et al., 2004; Loveday et al., 2012), alpha-lactalbumin (Goers et al., 2002; Otte et al., 2005), and kappa-casein (Ecroyd et al., 2008);
egg white proteins, such as lysozyme (Goda et al., 2000; Arnaudov and De Vries 2005), and ovalbumin (Pearce et al., 2007; Lara et al., 2012);
bovine serum albumin (Vetri et al., 2011; Arasteh et al., 2012);
haemoglobin (Jayawardena et al., 2017);
insulin (Kessler J., et al., 2017);
bovine and fish eye lens crystallin proteins (Garvey et al., 2009, Healy et al., 2012); and
some plant origin proteins like soy glycinin and soy beta-conglycinin (Akkermans et al., 2007; Tang and Wang 2010), kidney bean globulin (Tang et al., 2010); monellin from Serendipity berry (Konno et al., 1999); chickpea protein (Example 1 of the present application); and pea protein (Example 1 of the present application).

Accordingly, in one embodiment of the present invention, the fibrillar protein comprises an amyloid fibril and/or an amyloid-like fibril.

The proteinaceous component of the composition of the present invention may comprise a fibrillar protein and a non-fibrillar protein. The non-fibrillar protein may comprise native protein, intact protein, denatured protein, re-folded protein, protein aggregates, protein fragments, peptides, and/or combinations thereof. The proteinaceous component may comprise proteinaceous material derived from one or more protein sources. The fibrillar protein may be derived from a first protein source, and the non-fibrillar protein may be derived from a second protein source, wherein the first protein source and the second protein source are the same protein source or wherein the first protein and the second protein are different protein sources. The protein source may comprise a globular protein and/or a non-globular protein. In one embodiment, the proteinaceous component comprises proteinaceous material derived from a milk protein, a whey protein, a whey protein isolate, an egg protein, a bovine-derived protein, and/or a plant protein. Exemplary plant protein sources include a leguminous plant protein (including but not limited to a soy protein), a spinach protein, and/or a seaweed protein. Exemplary protein sources include, but are not limited to, beta-lactoglobulin, alpha-lactalbumin, kappa-casein, lysozyme, ovalbumin, bovine serum albumin, haemoglobin, insulin, bovine lens crystallin protein, fish eye lens crystallin protein, soy glycinin, soy beta-conglycinin, kidney bean globulin, rubisco, monellin, chickpea protein, pea protein, and/or any combination thereof.

In one embodiment of the present invention, the fibrillar protein of the composition of the present invention comprises a milk protein, a whey protein, a whey protein isolate, an egg protein, a bovine-derived protein, and/or a plant protein.

In one embodiment of the present invention, the fibrillar protein of the composition of the present invention comprises beta-lactoglobulin, alpha-lactalbumin, kappa-casein, lysozyme, ovalbumin, bovine serum albumin, haemoglobin, insulin, bovine lens crystallin protein, fish eye lens crystallin protein, soy glycinin, soy beta-conglycinin, kidney bean globulin, rubisco, monellin, and/or any combination thereof.

In one embodiment of the present invention, the fibrillar protein of the composition of the present invention comprises whey protein and/or soy protein. Suitably, the fibrillar protein comprises beta-lactoglobulin.

The composition of the present invention may comprise 0.1% to 20% w/v proteinaceous component. Suitably, the composition comprises 0.1% to 15% w/v proteinaceous component. More suitably, the composition comprises 0.5% to 10% w/v proteinaceous component. Even more suitably, the composition comprises 1% to 8% w/v proteinaceous component.

The proteinaceous component of the compositions of the present invention may comprise 5-95 wt % fibrillar protein. Suitably, the proteinaceous component comprises 10-80 wt % fibrillar protein. More suitably, the proteinaceous component comprises 20-70 wt % fibrillar protein. Even more suitably, the proteinaceous component comprises 30-60 wt % fibrillar protein.

The composition of the present invention may comprise a viscosity of 0.01 to 10 Poise. Suitably, the composition comprises a viscosity of 0.1 to 8 Poise. More suitably, the composition comprises a viscosity of 0.2 to 5 Poise. Even more suitably, the composition comprises a viscosity of 0.4 to 1 Poise.

The density of the composition of the present invention may be similar to that of water. Accordingly, the composition suitably comprises a density of 0.8 g/cm³ to 1.2 g/cm³. More suitably, the composition comprises a density of 0.9 g/cm³ to 1.1 g/cm³. Most suitably, the composition comprises a density of approximately 1.00 g/cm³.

The composition of the present invention may comprise a semi-solid state. Suitably, the composition of the present invention may comprise a gel, a foam, a film, a sheet, and/or a patch. More suitably, the composition of the present invention may comprise a gel. In one embodiment, the composition may comprise a thin gel. In another embodiment, the composition may comprise a thick gel. In yet another embodiment, the composition may comprise a fast fluid gel, a fluid gel, or a slow fluid gel.

The composition of the present invention may be dehydrated and/or lyophilised so as to form powders. These powders may be reconstituted to form compositions comprising protein fibrillar scaffolds. Accordingly in one aspect, the present invention comprises a composition in powder form, wherein the composition comprises a proteinaceous component, wherein upon addition of a solvent (including, but not limited to, water), the proteinaceous component of the composition comprises a scaffold of protein fibrils.

The composition of the present invention may exhibit shear thinning properties. Accordingly, in one embodiment the composition of the present invention may comprise a shear thinning composition. Suitably, the composition of the present invention comprises a thixotropic composition. More suitably, the composition of the present invention comprises a thixotropic gel.

The composition of the present invention may comprise water.

The composition of the present invention may comprise an emulsion. Suitably, the composition may comprise an oil in water emulsion. Alternatively, the composition may comprise an oil in water in oil emulsion. Suitable emulsions may comprise an oil including but not limited to a plant oil selected from the group consisting of a vegetable oil, a fruit oil, olive oil, avocado oil, canola oil, grapeseed oil, jojoba oil, almond oil, sweet almond oil, hazelnut oil, sunflower seed oil, coconut oil, argan oil, emu oil, tamanu oil, neem oil, apricot kernel oil, rose hip seed oil, evening primrose oil, castor oil and/or combinations thereof. Suitably, the composition may comprise 10% to 50% oil of the total volume. The composition may comprise an emulsion with a viscosity of 300 to 600 centipoise.

In one embodiment, the invention provides a composition comprising a proteinaceous component, wherein the proteinaceous component comprises 5-50 wt % fibrillar protein, wherein the fibrillar protein comprises a scaffold, wherein the fibrillar protein is derived from whey protein and/or soy protein, and wherein the composition comprises a shear thinning gel. Suitably, the proteinaceous component comprises 10-40 wt % fibrillar protein.

### Attachment and Extended Release Properties

The present invention is based, at least in part, on the unexpected discovery that a protein fibril scaffold permits or provides for the attachment of a wide range of agents and permits or provides for the extended release of the agents.

The attachment and extended release of agents having different properties is desirable, and without wishing to be bound by theory, it is believed that the large particle size, high surface area, and unique hydrophilic and lipophilic surface composition of the fibrillar scaffold provides it with its attachment and release properties. The surface composition of the fibrillar scaffold is due in part to the amino acids within the fibrils, and in particular their characteristics which are exposed upon formation of the scaffold, and are thus available for interaction with an agent. The amino acid characteristics include the side chains and/or groups, where the side chains can be charged, polar or hydrophobic, and the groups can be acidic (carboxyl group) or basic (amino group). The exposure of these different side chains and/or groups thus permits the attachment and release of agents having different properties, such as hydrophilic, hydrophobic, and/or lipophilic agents. The attachment of an agent to the fibrillar scaffold may be permitted in any suitable interaction, including by way of covalent and/or non-covalent bonding either directly or indirectly such as via a linker. The nature of the protein fibril scaffolds is such that more than one side chain and/or more than one group can be exposed in a fibrillar scaffold, permitting the attachment and release of agents having different properties. For instance, the composition of the present invention may be suitable to receive a hydrophilic agent and a lipophilic agent. The compositions of the present invention may suitably comprise universal vehicles.

Accordingly, in one aspect the composition of the present invention is suitable for attachment to an agent. The composition of the present invention may further be suitable for extended release of the agent.

In a further aspect, the composition of the present invention provides for attachment of an agent. The composition of the present invention may further provide for extended release of the agent.

In another aspect, the composition of the present invention permits the attachment of an agent. The composition of the present invention may further permit the extended release of the agent.

In another aspect the composition of the present invention further comprises an agent.

As used herein, the phrases “attach an agent”, “attachment of an agent”. “provides for attachment of an agent”, “permits the attachment of an agent”, and the like, refers to a property of the fibrillar protein scaffold to interact with an agent such that they are attached in any suitable way, including by direct or indirect attachment, including by way of being bound (covalently or non-covalently), and/or attached via a linked, and/or otherwise attached to each other.

In one aspect the present invention provides a composition comprising a proteinaceous component and an agent, wherein the proteinaceous component comprises a fibrillar protein scaffold. In another aspect the present invention provides a composition comprising a proteinaceous component and an agent, wherein the proteinaceous component comprises a fibrillar protein scaffold, and wherein the fibrillar protein scaffold and the agent are attached to each other. In yet another aspect, the present invention provides a composition comprising a proteinaceous component and an agent, wherein the proteinaceous component comprises a fibrillar protein scaffold, and wherein the fibrillar protein scaffold and the agent are bound to each other. In yet another aspect, the present invention provides a composition comprising a proteinaceous component and an agent, wherein the proteinaceous component comprises a fibrillar protein scaffold, and wherein the fibrillar protein scaffold and the agent are linked to each other.

As used herein, the phrases “releases an agent”, “release of an agent”, “extended release of an agent”, “provides for release of an agent”, “provides for extended release of an agent”, “permits the release of an agent”, “permits the extended release of an agent”, and the like, refers to the property of the fibrillar protein scaffold to provide for an extended release of an attached agent. The term “extended release” refers to the prolonged release of an agent from the fibrillar protein scaffold, and is intended to encompass the following terms: “extended release”, “sustained release”, “prolonged release”, “controlled release”, “delayed release”, “timed release”, and/or “time release”.

### Applications

The development of compositions that will receive agents and provide for extended release of agents is desirable, in particular where a composition is capable of receiving and releasing a wide range of agents, as that provides the user with a high degree of flexibility to customise the composition to the user's requirements.

For instance, the composition of the present invention may be formulated for topical administration to a keratinaceous surface, including but not limited to skin, hair, and nail. The composition of the present invention may thus comprise a skincare composition, sunscreen composition. As used herein, the term “skin care” or the like, is intended to encompass care of any keratinaceous surface, including skin, hair, and/or nail, and accordingly the term “skincare composition” (and the like) is intended to encompass compositions for the care of any keratinaceous surface, including skin, hair, and/or nail. Details of these aspects of the present invention are described further herein.

The composition of the present invention may be formulated for administration to a wound. The composition of the present invention may thus comprise a wound care composition.

The compositions of the present invention provide for the receipt and extended release of a wide range of agents, and this is desirable for the user. For instance, the ability of the compositions to receive a wide range of agents provides for a user to select an agent for use in the composition which exhibits the most desirable biological properties for the intended use of the composition. The ability of the compositions to provide for extended release of the agent provides for the agent to be released over a longer period. For instance, when compared to a composition that provides for immediate release of an agent, the compositions of the present invention may provide the intended use, benefit or activity of the agent for a prolonged period. This means the compositions may have their desired effect for a longer period of time. The extended release property of the compositions may also be employed to avoid undesirable side effects from the agent as an extended release of the agent may permit the release of smaller amounts (e.g., lower dosages, smaller volumes) of the agent over a time period compared to an immediate bolus release of the agent. Undesirable side effects in the context of topical administration to a keratinaceous surface or a wound, include, for instance irritation or allergy.

The term “agent” as used herein refers to any suitable agent. The agent may comprise a biologically active agent. Suitably, the composition may comprise an agent suitable for skin care, sunscreen, and/or wound care. Suitable and exemplary agents are discussed in detail below.

### Skincare Compositions

In developing the compositions of the present invention, the inventors discovered that the compositions possess especially desirable properties for skin care applications. As already mentioned herein, the compositions of the present invention provide for the attachment and extended release of a wide range agents with different properties so that a skincare composition may comprise one or more agents that is/are desirable in the use of skin care. There are many agents of many different properties that are desirable to be used in skin care, depending on the benefits sought. Furthermore, the ability of the compositions to provide the extended release of retained agents is desirable in skin care applications so that a composition may provide the benefit of a biologically active agent over a longer period time compared with a composition that provides for immediate release of the agent, and furthermore may provide benefits of smaller amounts of the agent being released at one time point, as discussed above. The compositions may also comprise emulsions.

Additionally, the attractiveness of the compositions of the present invention for skincare applications may be due to other properties of the compositions. For instance, the compositions of the present invention can comprise a semi-solid form, such as a gel or a film, and in particular the composition of the present invention my comprise a shear thinning gel including but not limited to a thixotropic gel. Thixotropic gels are especially desirable for use in some skincare applications as they are readily applied to keratinaceous surfaces (the shear transforms the gel to a spreadable consistency) but revert to a more solid-like form upon storage so that they are easier to store and handle. The compositions of the present invention were also found to be safe for skin as they are non-irritants for skin and are also non-phototoxic. Additionally, the compositions were shown to have skin adhesion properties suitable for application in skin care. These skin adhesion properties can be applied in a skincare composition in a number of ways, for instance they can have application by trapping moisture in the skin and preventing the moisture from escaping and/or by acting as a barrier to prevent or reduce the impact of external factors such as air pollutants or wind on damaging the skin. The compositions of the present invention also break down naturally, leaving no residue on skin or in the environment.

The composition of the present invention may thus be formulated for topical administration to a keratinaceous surfaces, including but not limited to skin, hair, and/or nails. Accordingly, in one aspect, the composition of the present invention may comprise a skincare composition. Suitably, the skincare composition comprises an agent. In another aspect, the present invention comprises a composition for use in skin care. Suitably, the composition comprises an agent.

Another aspect of the present invention provides a skincare composition comprising a fibrillar protein scaffold. Suitably, the skincare composition comprises an agent. Yet another aspect of the present invention provides a composition comprising a fibrillar protein scaffold for use in skin care. Suitably, the composition comprises an agent.

In another aspect of the present invention, there is provided the use of a fibrillar protein scaffold in the manufacture of a skincare composition. In yet another aspect of the present invention, there is provided the use of a fibrillar protein scaffold in the manufacture of a composition for use in skin care.

Yet another aspect of the present invention provides a method of caring for a keratinaceous surface in a subject, wherein the method comprises administering a composition of the present invention to the keratinaceous surface of the subject. Suitably, the composition comprises a fibrillar protein scaffold and an agent. In some embodiments, the keratinaceous surface comprises skin, hair, and/or nail.

As used herein, the term “skin care”, or the like, is used with reference to any keratinaceous surface, including skin, hair, and/or nail, and accordingly the term “skincare composition” (and the like) is intended to encompass compositions for the care of any keratinaceous surface, including skin, hair, and/or nail. The term “care” for example as used in the phrase “skin care”, and in the phrase “method of caring for a keratinaceous surface” and similar, refers to the prevention, treatment, or management of a keratinaceous surface, wherein: “prevention” refers to the prevention or inhibition of the onset, recurrence, and/or development of a disease or condition or a symptom thereof in a subject resulting from the administration of a composition; “treatment” refers to the reduction, amelioration or inhibition of the severity, progression and/or duration of a disease or condition, and/or a symptom of a disease or condition resulting in a subject from the administration of a composition; and “management” refers to the beneficial effects derived from administration of a composition, while not resulting in a cure of the disease or condition resulting in a subject from the administration of a composition.

Suitable skincare compositions in accordance with the present invention include but not limited to a cleanser, a moisturiser, a foundation, a day cream, a night cream, a body lotion, a hand cream, an eye cream, a lotion, a beauty product, a cosmetic, a cosmeceutical, a shaving cream, a shaving gel, a hair cream, a hair gel, a hair mousse, a hair treatment product (including, but not limited to a leave-in conditioner), and/or a hair serum.

The composition of the present invention may comprise an agent. When the composition of the present invention comprises a skincare composition or is used for skin care, any agent suitable for skin care may be employed. Suitable agents include, but are not limited to a moisturising agent, a hydrating agent, a lipid, an oil, a vitamin, a mineral, a plant extract, a herbal extract, an anti-acne agent, and/or an agent providing a health benefit. Exemplary agents include, but are not limited to, vitamin A, retinol, retinyl palmitate, retinaldehyde, vitamin C, ascorbic acid, vitamin E, tocopherol, alpha tocopherol, hyaluronic acid, hyaluronan, sodium hyaluronate, collagen, keratin, plant oil, vegetable oil, fruit oil, olive oil, avocado oil, canola oil, grapeseed oil, jojoba oil, almond oil, sweet almond oil, hazelnut oil, sunflower seed oil, coconut oil, argan oil, emu oil, tamanu oil, neem oil, apricot kernel oil, rose hip seed oil, evening primrose oil, castor oil, glycerin, vegetable glycerin, bee venom, a bee venom derivative, honey, Manuka honey, a beta-hydroxy acid, salicylic acid, capric/caprylic triglyceride, caffeine, alpha-hydroxy acid, glycolic acid, lactic acid, fruit acid, cetyl alcohol, stearyl alcohol, and/or cetearyl alcohol. The composition of the present invention may further comprise an agent suitable for the formulation of a skincare composition. Suitable agents include, but are not limited to, a buffer, an acid and/or a base (including but not limited to sodium hydroxide, lactic acid), vegetable glycerin, an emulsifier, a thickener, a preservative, a lubricant, a pigment, and/or a fragrance.

### Suncare Compositions

For the reasons described above with regards to skincare compositions, it was also recognised by the inventors that the compositions of the present invention are suitable for use in suncare applications. For instance, the adhesion properties of the compositions is especially desirable for a suncare composition which comprises an agent to shield the skin from ultraviolet radiation, and it was shown by the inventors that compositions of the present invention comprising ultraviolet blockage agents provided greater ultraviolet blockage after washing on a skin mimetic when compared to commercially available suncare compositions containing ultraviolet blockage agents, and bind ultraviolet blockage agents for longer compared to control test agents.

Thus in yet another aspect, the composition of the present invention may comprise a suncare composition. Suitably, the suncare composition comprises an agent. In another aspect, the present invention comprises a composition for use in sun care. Suitably, the composition comprises an agent.

Another aspect of the present invention provides a suncare composition comprising a fibrillar protein scaffold. Suitably, the suncare composition comprises an agent. Yet another aspect of the present invention provides a composition comprising a fibrillar protein scaffold for use in sun care. Suitably, the composition comprises an agent.

In another aspect of the present invention, there is provided the use of a fibrillar protein scaffold in the manufacture of a suncare composition. In yet another aspect of the present invention, there is provided the use of a fibrillar protein scaffold in the manufacture of a composition for use in sun care.

Yet another aspect of the present invention provides a method of protecting a keratinaceous surface in a subject from UV radiation, wherein the method comprises administering a composition of the present invention to the keratinaceous surface of the subject. Suitably, the composition comprises a fibrillar protein scaffold and an agent. In some embodiments, the keratinaceous surface comprises skin, hair, and/or nail.

Suitable compositions include, but are not limited to, a suncare composition, a sunblock, a sunscreen, a suntan composition, and/or a suntan lotion.

Accordingly, as a suncare composition, the composition of the present invention may comprise an agent which blocks, reduces or minimises the absorbance of ultraviolet radiation by the skin. Suitable agents may be selected from the group consisting of a zinc-containing agent such as zinc oxide, a titanium-containing agent such as titanium oxide, avobenzone, a plant extract (including but not limited to an extract of Gingko biloba, silymarin, an extract of pomegranate, an extract of Polypodium leucotomos, an extract of peach, an extract of moldavian dragonhead, an extract of Viola tricolor, an extract of carrot, carrot seed oil, wheat germ oil, an extract of Symplocos racemosa, aloe vera, an extract of aloe vera, an extract of basil, tumeric, and/or an essential oil), propolis, curcumin, butyl methoxydibenzoylmethane, and/or any agent identified above as being suitable for skincare compositions. Suitably, the composition comprises zinc oxide at less than 20% w/w, more suitable less than 15% w/w. The zinc oxide may comprise micronised zinc oxide.

The composition of the present invention may comprise a sun protection factor (SPF) of 15, more suitably 15+, even more suitably 30, most suitably 30+.

The composition of the present invention may comprise a skincare composition and a sun care composition.

### Wound Care Compositions

The present inventors have also recognised the application of the compositions of the present invention in wound care applications. For instance, the composition of the present invention can comprise a semi-solid form, such as a gel or a film. The compositions of the present invention were also found to be safe for skin as they are non-irritants for skin and are also non-phototoxic. Additionally, the skin adhesion properties of the compositions make them suitable for application in wound care, for instance by absorbing water from within the wound. The resistance to degradation by protease enzymes of the protein fibrils in the composition, such as would be found in wounds, also permits the composition of the present invention to maintain its hydration and delivery properties when applied to wounds. The ability of the compositions to provide the extended release of agents is also desirable in wound care applications, where cleaning (e.g. antimicrobial) and healing agents can be incorporated into the compositions. Furthermore, experiments conducted by the inventors demonstrated that the protein compositions selectively inhibits undesirable matrix metalloproteinases (MMPs), which are enzymes that can be found in wounds but does not inhibit desirable MMPs. In developing the present invention, the inventors discovered that the protein compositions of the present invention inhibit MMP2 and MMP9, both of which are known to cause damage in wounds (they play a role in inflammation), but did not inhibition MMP1, a collagenase is essential for remodelling tissue as it heals.

Accordingly in yet another aspect, the present invention provides a wound care composition. Suitably, the wound care composition comprises an agent. In another aspect, the present invention comprises a composition for use in wound care. Suitably, the composition comprises an agent.

Another aspect of the present invention provides a wound care composition comprising a fibrillar protein scaffold. Suitably, the wound care composition comprises an agent. Yet another aspect of the present invention provides a composition comprising a fibrillar protein scaffold for use in wound care. Suitably, the composition comprises an agent.

In another aspect of the present invention, there is provided the use of a fibrillar protein scaffold in the manufacture of a wound care composition. In yet another aspect of the present invention, there is provided the use of a fibrillar protein scaffold in the manufacture of a composition for use in wound care.

Yet another aspect of the present invention provides a method of protecting or treating a wound in a subject, wherein the method comprises administering a composition of the present invention to the wound of the subject. Suitably, the composition comprises a fibrillar protein scaffold and an agent.

Another aspect of the present invention thus provides a method of preventing and/or treating an infection, and/or for preventing and/or reducing inflammation, and/or for promoting or stimulating wound healing, wherein the method comprises administering a composition of the present invention to the wound of the subject. Suitably, the composition comprises a fibrillar protein scaffold and an agent.

Suitable wound care compositions include, but are not limited to gels, creams, lotions, foams, films, bandages, tape, wrap, strapping, adhesives, and/or plasters.

Accordingly, as a wound care composition, the composition of the present invention may comprise an agent for preventing and/or treating an infection, and/or for preventing and/or reducing inflammation, and/or for promoting or stimulating wound healing. The agent may thus suitably comprise an antibiotic, an antimicrobial, silver, an antimicrobial peptide, a matrix protein, collagen, a growth factor, a vitamin, vitamin A, vitamin C, vitamin E, an alginate, a hydrocolloid, a hydrogel, and/or a nonsteroidal anti-inflammatory drug (NSAID).

The invention is further described with reference to the following examples. It will be appreciated that the invention as claimed is not intended to be limited in any way by these examples.

### EXAMPLES

### Example 1: Protein Compositions—Manufacturing & General Properties

### Materials and Methods for Making the Protein Compositions

Three protein compositions (protein composition A, protein composition B, and protein composition C) were made from whey protein isolate (WPI) using the following manufacturing process.

A solution of WPI at 8% w/w was prepared in a reaction chamber with the pH adjusted to 2 using phosphoric acid. The solution in the reaction chamber was then heated at 80° C. with agitation and the pH re-adjusted back to 2 if necessary. The solution was then cooled to room temperature. Material was then harvested from the reaction chamber into sealed containers, and stored at 4° C. until needed.

The protein compositions were shown to comprise protein fibrils in scaffold form, as further characterised below.

Using the same method as described above, protein fibril scaffolds were made from soy protein, chickpea protein, and pea protein.

### Characterisation of the Proteinaceous Component

Characterisation of the protein compositions revealed that the proteinaceous component of the compositions was composed of fibrillar and non-fibrillar (including native and denatured protein). The level of fibrillar protein was determined based on a comparison of thioflavin T fluorescence between soluble and insoluble components where it was determined that the total fibril content in the proteinaceous component of protein composition B was 27% and the total fibril content in the proteinaceous component of protein composition C was 37%.

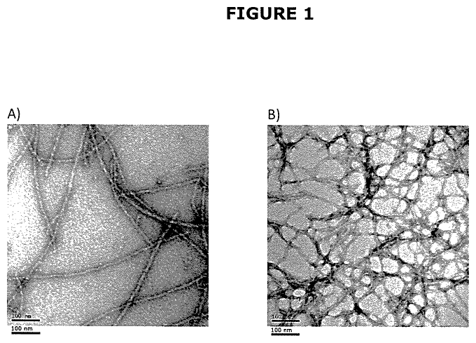

The protein fibrils comprised amyloid-like protein fibrils produced primarily but not exclusively from beta-lactoglobulin. Transmission electron microscopy (TEM) analysis showed the fibrils to comprise long, unbranched, rod-shaped structures ranging from 9 to 50 nm across, and up to 1 μm long in scaffold form. FIG. 1 shows some TEM imagery obtained: where A) is protein composition A; and B) is protein composition C. The protein fibrils in scaffold form can be clearly identified in each.

The protein fibrils were shown to be non-water soluble, slow-degrading and partially resistant to proteolysis.

### General Properties

The protein compositions A, B, and C, each comprised a semi-solid material in the form of a gel. The total protein content in the compositions ranged from 3 to 9% w/v denatured whey protein isolate. The viscosities and densities of the protein compositions were measured, where density=M (g)/V (cm³), where M=Mass and V=Volume (10 mL). The viscosities of the gels increased proportional to protein concentration. The densities of the protein compositions were like that of water. The properties of the protein compositions are shown in Table 2:

TABLE 2
Properties of protein compositions

| | | Viscosity | Density |
|---|---|---|---|
| | Protein content | (Poise) | (g/cm³) |
| Protein composition A | 3-5% (whey protein) | 0.4 ± 0.5 | 1.02 |
| Protein composition B | 5-7% (whey protein) | 2.5 ± 1 | 1.00 |
| Protein composition C | 7-9% (whey protein) | 5.5 ± 1 | 1.03 |

The appearances of the protein compositions was also assessed and the results are shown in Table 3:

TABLE 3
Appearance of protein compositions

| | Consistency | Colour | Texture |
|---|---|---|---|
| Protein composition A | Thin, fast fluid | Light olive | Watery |
| | to fluid gel | green | |
| Protein composition B | Thick, fluid gel | Medium olive | Silky |
| | | green | |
| Protein composition C | Thick, slow | Dark olive | Silky |
| | fluid gel | green | |

The protein compositions had a sweet, slightly sulphurous smell with custard notes. Upon addition of a preservative agent (Geogard 221), the protein compositions gave an almond-like (marzipan) smell.

The protein compositions were also discovered to break down naturally, leaving no residue, both when applied to the skin or in the environment (data not shown).

### Formation of Emulsions

The protein compositions were also shown to form oil in water (o/w) emulsions without needing to be heated and without the need of emulsifying agents. It was shown that these cold-process o/w emulsions can be made with various vegetable oils (including olive oil) ranging from 10% to 30% in content. Furthermore, the o/w emulsion made from 20% oil could be diluted a further 2-fold with oil. The viscosities of the o/w emulsions made from protein composition B and protein composition C ranged from 300 to 600 cP.

The protein compositions were also shown to form o/w emulsions using conventional processing methods and emulsifiers. Furthermore, it was shown that the protein compositions could also be used to form oil in water in oil (o/w/o) emulsions also using conventional processing methods and emulsifiers, where these emulsions exhibited a higher level of water resistance and a subsequently higher content of oil (up to 50% of the total volume).

Protein composition B and protein composition C were also shown to be capable of swelling and absorbing up to 25% of their weight in water.

### Shear Thinning, Thixotropic Properties

The viscosities of the protein compositions did not change over time when measured at 21° C. or 30° C., and when not subjected to shear (data not shown).

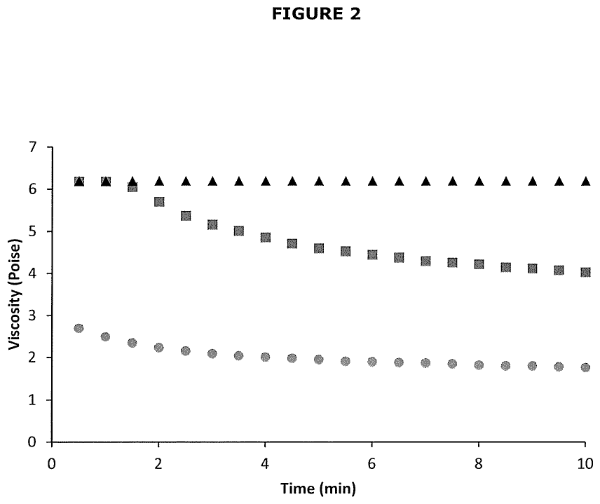

When subjected to shear, the protein compositions were shown to comprise shear thinning, thixotropic gels. For instance, gel viscosities of protein composition B, protein composition C and a Newtonian liquid were measured under shear stress at 21° C. with Brookfield DV3T, spindle SC4-21, at 100 rpm, and the results are shown in FIG. 2, where: the dark triangles (▴) represent the Newtonian liquid; the grey squares ( [symbol] ) represent protein composition C; and the grey circles ( [symbol] ) represent protein composition B. As can be seen, the two protein compositions B and C demonstrated shear thinning, as the viscosity of the compositions decreased over time, while the viscosity of the Newtonian liquid did not.

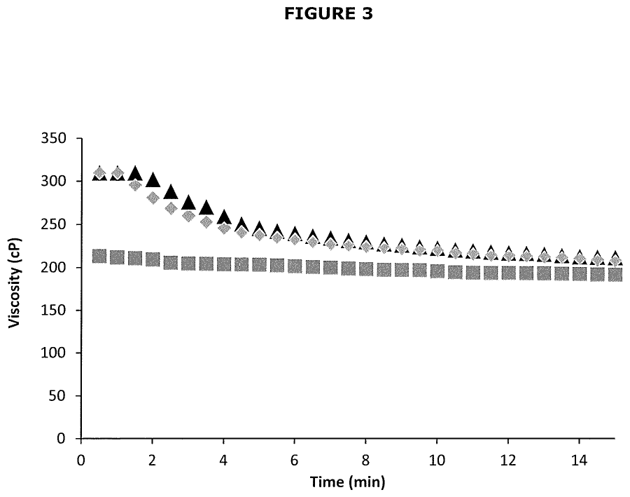

After becoming fluid post shearing, the protein compositions were also shown to regain a semi-solid form upon standing, undisturbed for 1 day, as shown for protein composition C in FIG. 3 (measured at 21° C. using Brookfield DV3T, spindle SC4-21, at 200 rpm), where: the dark triangles (▴) represent the initial viscosity profile of the protein composition C; the grey squares ( [symbol] ) represent the viscosity profile of protein composition C after 1 minute rest; and the grey diamonds ( [symbol] ) represent the viscosity profile of protein composition C after 24-hours rest.

For FIGS. 2 and 3, the x-axis shows the time that the protein composition or Newtonian liquid remained inside the viscometer.

### Films, Foams, Sheets & Powder Forms

The compositions made in accordance with the method described above were also converted into films, foams, sheets and powder forms.

The compositions were also converted into powder form by way of lyophilisation (freeze drying) or other drying. The powders could be reconstituted with the addition of water (and optional adjustment of salt levels if required) to resemble those of the compositions prior to lyophilisation.

### Example 2: Protein Compositions—Universal Vehicles Capable of Extended Release

Protein compositions A, B, and C produced in Example 1 were shown to be able to be diluted in water or PBS, with a dilution factor of up to an additional 100% of their original volume.

Furthermore, the protein compositions A, B, and C produced in Example 1 were found to be universal vehicles, exhibiting the property of carriers for both hydrophilic and hydrophobic agents. Table 4 shows the loading capacity of protein composition C with various agents:

TABLE 4
Loading capacity of protein composition C with various
hydrophilic and hydrophobic agents at room temperature

| Agent | Type | Loading | | Solubility (water) | |
|---|---|---|---|---|---|
| Caffeine | Hydrophilic | 350 | mg/g* | 20 | mg/mL |
| Ascorbic acid | Hydrophilic | 350 | mg/g* | 330 | mg/mL |
| Tryptophan | Hydrophilic | 350 | mg/g* | 11.4 | mg/mL |
| Quinine sulphate | Hydrophobic | 10 | mg/g** | Slightly soluble | |
| Benzoic acid | Hydrophobic | 10 | mg/g** | 3.44 | mg/mL |
| Retinal | Hydrophobic | 1 | mg/g** | Nearly insoluble | |
| Retinyl palmitate | Hydrophobic | 25 | mg/g** | Insoluble | |
| Methylene blue | Hydrophobic | 10 | mg/g** | 40 | mg/mL |

*Maximum loading;
**Loading trial to date, not maximum loading

The protein compositions were found to adsorb and release agents, where the release of agents occurred gradually over time. This extended release property of the protein compositions was further assessed using various agents and compared with solutions of an equivalent protein concentration of whey protein isolate (WPI) as shown in Table 5:

TABLE 5
Protein concentrations of the tested protein compositions
and the WPI solutions used for comparison

| | WPI solution for | Protein |
|---|---|---|
| Protein compositions | comparison | concentration |
| Protein composition A | WPI100 | 10 mg/mL |
| Protein composition B | WPI200 | 40 mg/mL |
| Protein composition C | WPI300 | 80 mg/mL |

One agent tested was quinine sulphate (QS).

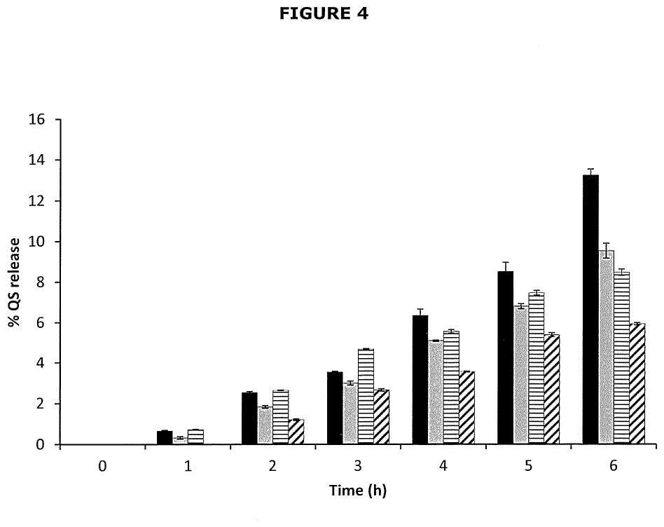

A significant difference in release profiles of quinine sulphate (QS) for protein composition A and protein composition B compared to their counterpart WPI solutions of equivalent protein concentrations was observed, as shown in FIG. 4, where it can be seen that the protein compositions demonstrated a delayed release compared to their counterpart WPI solutions.

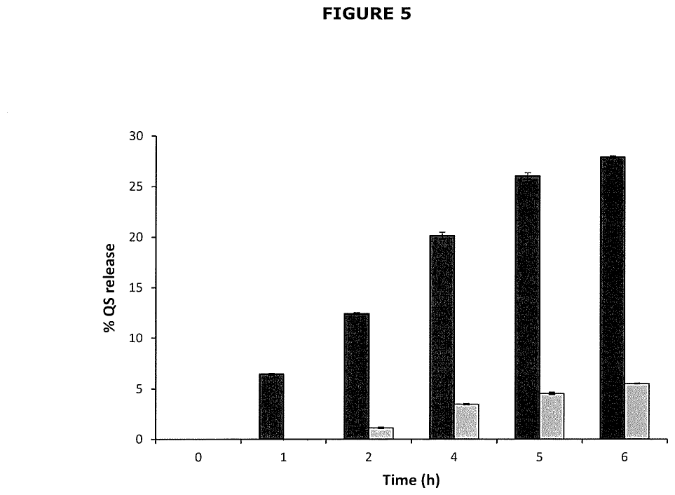

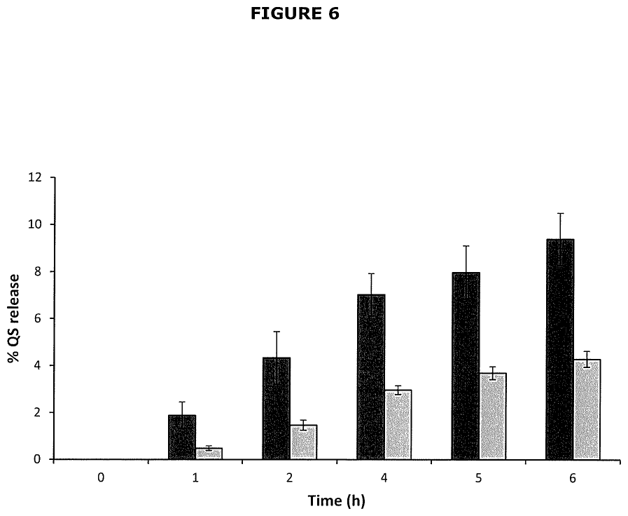

Protein composition C demonstrated a 4-fold delay in the release of quinine sulphate (QS), with 6% of quinine sulphate released in 6 hours, compared to the WPI solution of equivalent protein concentration with 28% of quinine sulphate released in 6 hours, as shown in FIG. 5. In FIG. 5, the grey (lighter) bars represent the % of quinine sulphate released from protein composition C and the black (darker) bars represent the % of quinine sulphate released from the WPI solution (% QS release refers to the percentage of quinine sulphate released over time). Protein composition C also demonstrated a 2-fold delay in release of quinine sulphate (QS) with 15% released in 24 hours, compared to the commercially available gel sodium gluconate with 29% quinine sulphate released in 24 hours, as shown in FIG. 6. In FIG. 6, the grey (lighter) bars represent the % of quinine sulphate released from protein composition C and the black (darker) bars represent the % of quinine sulphate released from the commercially available gel (% QS release refers to the percentage of quinine sulphate released over time).

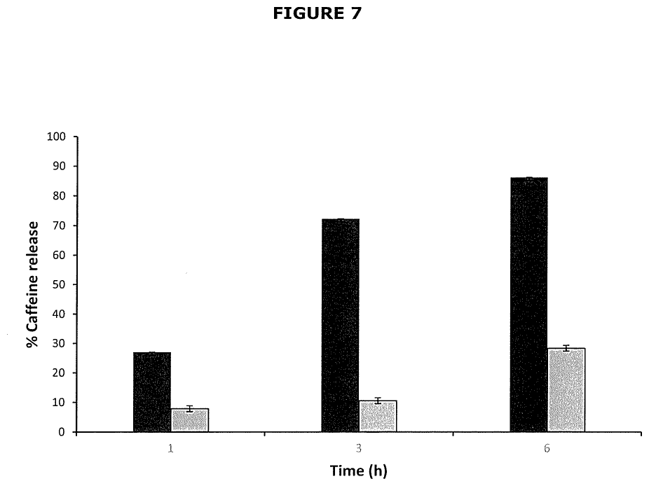

Further experimental work with another agent (caffeine) was performed to ascertain differences in the extended release profiles of the protein compositions due to the hydrophobicity and size of the agent. Quinine sulphate has a molecular weight of 360.9 g/mol and a log P of 2.48. Caffeine has a molecular weight of 194.19 g/mol and a log P of −0.8. Protein composition C was found to have a 3-fold delay (FIG. 7) in release of caffeine (28% caffeine released in 6 hours) compared to a PBS solution (86% caffeine released in 6 hours). In FIG. 7, the grey (lighter) bars represent the % of caffeine released from protein composition C and the black (darker) lines represent the % of caffeine released from the PBS buffer. However, caffeine was released 4.6-fold faster from protein composition C than the rate of release of quinine sulphate from protein composition C (6% quinine sulphate released in 6 hours, FIG. 5; 28% caffeine release in 6 hours, FIG. 7).

It was thus deemed that the protein compositions release lipophilic compounds more slowly than hydrophilic compounds.

### Zinc Oxide Interaction

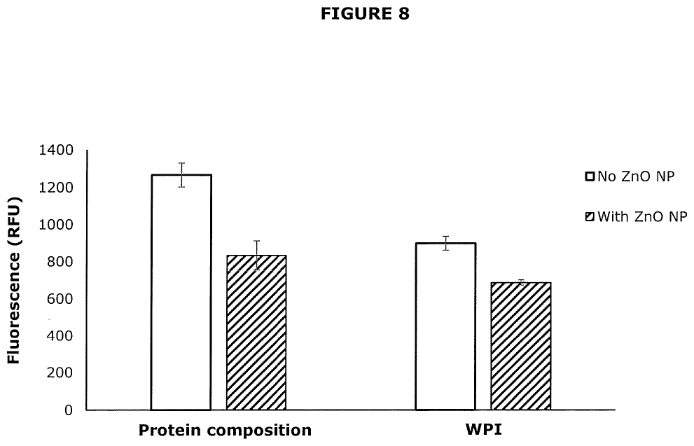

It is known that when zinc oxide nanoparticles (ZnO NPs) interact with protein, the fluorescence intensity of the protein decreases and that this can be measured (Kathiravan et al., 2009 and Bhogale et al., 2013). ZnO NPs were found to interact with the protein composition C produced in Example 1, whereby the fluorescence spectra of the protein compositions and of whey protein isolate (WPI, positive control) were recorded in the presence and absence of ZnO NPs, as shown in FIG. 8. In FIG. 8, a decrease in the fluorescence intensity for both the protein composition (Δ431 RFU) and the WPI control (Δ210 RFU) upon exposure to the ZnO NPs was observed, indicating that both the protein composition and the WPI control can bind ZnO NPs.

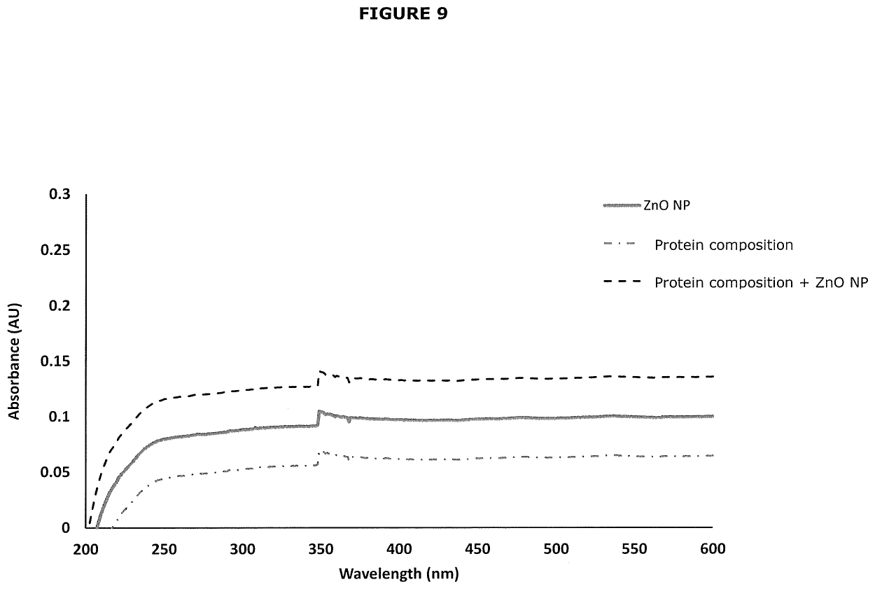

To verify that the decrease in fluorescence intensity in the presence of ZnO NPs was not due to a precipitation effect upon complex formation, the absorbance spectra of the protein composition in the presence of ZnO NPs and in the absence of ZnO NPs was recorded. The results are shown in FIG. 9, where the absorbance spectra of the protein composition in the absence (dashed grey line) and presence (dashed black line) of ZnO NPs (solid grey line) is shown. An increase in the absorbance signal of protein composition in the presence of ZnO NPs is shown, showing that the complex of the ZnO NPs and the protein composition has not precipitated out of solution.

### Avobenzone Interaction

The diffusion rate of avobonezone from an emulsion, in the presence and absence of protein composition C produced in Example 1, was measured. The results are shown in

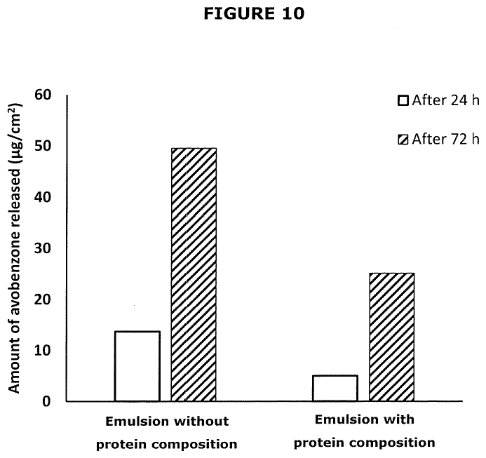

FIG. 10, where it can be observed that an emulsion of avobenzone without the protein composition diffused 2.7 times faster than an emulsion of avobenzone with the protein composition. Thus, it can be concluded that the protein compositions bind molecules tighter than an emulsion alone.

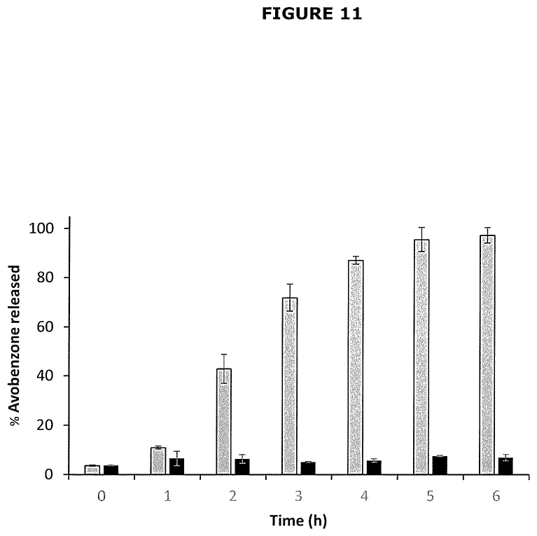

Protein composition C exhibited a delayed release of avobenzone compared to an oil solution containing avobenzone, as shown in FIG. 11. In FIG. 11, the darker bars represent the % of avobenzone released from protein composition C and the lighter bars represent the % of avobenzone released from the oil solution.

### Example 3: Protein Compositions—Suitability for Use

### Safety

An in vitro skin irritation assay for predicting dermal irritation was conducted following the OECD 439 protocol entitled: “In-vitro Skin Irritation: Reconstructed Human Epidermis Test Method” using the EpiDerm™ Model (MatTek, Ashland, Mass., USA).

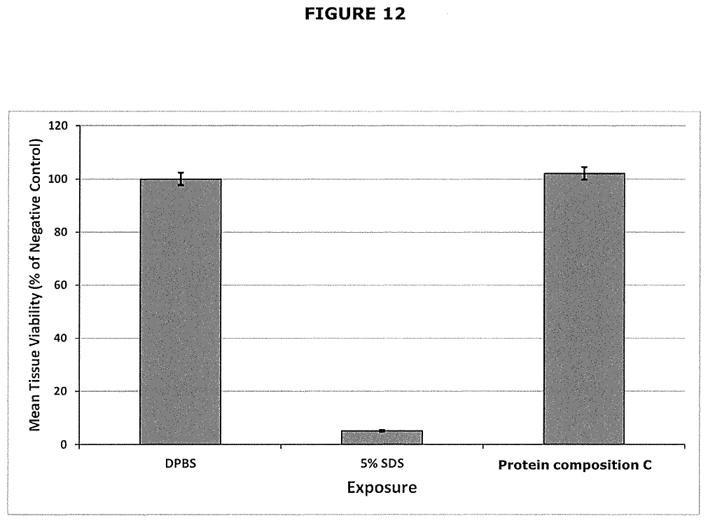

This EpiDerm™ model is used to assess the potential dermal irritation of a test article by determining the viability of the tissues via MU assay following exposure to the test articles (here: the protein compositions produced as in Example 1). Pre-testing showed that the protein compositions were not coloured nor did they auto-reduce MU. Tissues were then exposed to the protein compositions and controls for one hour, followed by a 42-hour post-exposure recovery period. The viability of each tissue was then determined by MU assay with the finding that the protein compositions were non-irritants. The result for the test of protein composition C is shown in FIG. 12, where the mean tissue viability (% of negative control) is shown for the negative control (DPBS), the positive control (5% SDS), and protein composition C.

An in vivo 24-hour patch test was then performed and confirmed the findings of the in vitro skin irritation results. In brief, protein composition C (produced as in Example 1) was applied on the backs of 50 human volunteers and its potential to cause an area of erythema and edema at the site of application after 24-hours of exposure was determined. The protein composition was found to be a non-primary irritant to the skin.

An in vitro assay to determine the potential to cause phototoxicity was also conducted on protein composition C (produced as in Example 1), using the OECD 432 protocol entitled: “In Vitro 3T3 NRU Phototoxicity Test”, and protein composition C was found to be negative for causing phototoxicity. The results are shown in Table 6, and it is noted that the controls responded as expected in this experiment.

TABLE 6
Phototoxicity results for protein composition C

| | Cell Viability IC50 | | | | |
|---|---|---|---|---|---|
| | Minus UVA | Plus UVA | Mean | Mean | |
| Test article | (μg/mL) | (μg/mL) | PIF | MPE | Comment |
| Histidine | >1000 | >1000 | 1.0 | −0.016 | Non- |
| | | | | | phototoxic |
| Chlorpromazine | 7.0 | 0.1 | 70 | 0.510 | Phototoxic |
| Protein | >125 | >125 | 1.0 | −0.030 | Non- |
| composition C | | | | | phototoxic |

### Adhesion to Keratinaceous Surfaces

A water resistance assay was carried out to determine the ability of the protein compositions to adhere to keratinaceous surfaces, including skin, hair, or nails. Vitro-Skin™ (IMS Inc, Portland, Me., USA), is a testing substrate that mimics the surface properties of human skin. It has been formulated to have topography, pH, critical surface tension and ionic strength that is similar to human skin. The substrate can be used to perform in-vitro SPF/UVA testing and in-vitro water resistance SPF/UVA testing.

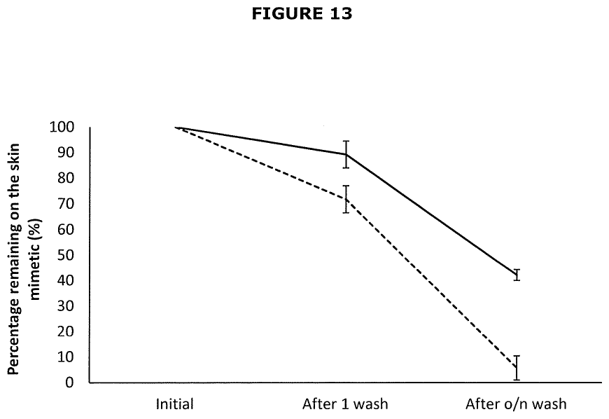

The protein composition C produced in Example 1 and whey protein isolate (WPI) were separately applied at an amount of 2 mg/cm² onto 12.25 cm² pieces of Vitro-Skin and subjected to a 2 wash cycle in water. Each wash cycle consisted of submersion of the skin sample into 650 mL of water, stirred at 200 rpm at 34° C. for 20 minutes and overnight. UV spectrophotometric analysis at 280 nm (lambda max of protein) was used to measure the amount of protein on the membrane before and after washing, and these results are shown in FIG. 13, where the solid line represents the amount of the protein composition and the dashed line represents the amount of the WPI retained on the skin mimetic after 1 wash (“After 1 wash”) and after overnight wash (“After o/n wash”). The results show that a greater amount of the protein composition was retained on the skin mimetic compared to the WPI (protein control).

A similar assay was performed to compare the water resistance of four compositions containing zinc oxide (ZnO). The four compositions comprised:
commercially available sunscreen formulation X with 24.5% zinc oxide;
commercially available sunscreen formulation Y with 15% zinc oxide;
protein composition C containing whey protein manufactured as described in Example 1 with 15% zinc oxide (ZnO); and
a protein composition containing soy protein manufactured using the method described in Example 1 with 15% zinc oxide (ZnO) and 3% avobenzone.

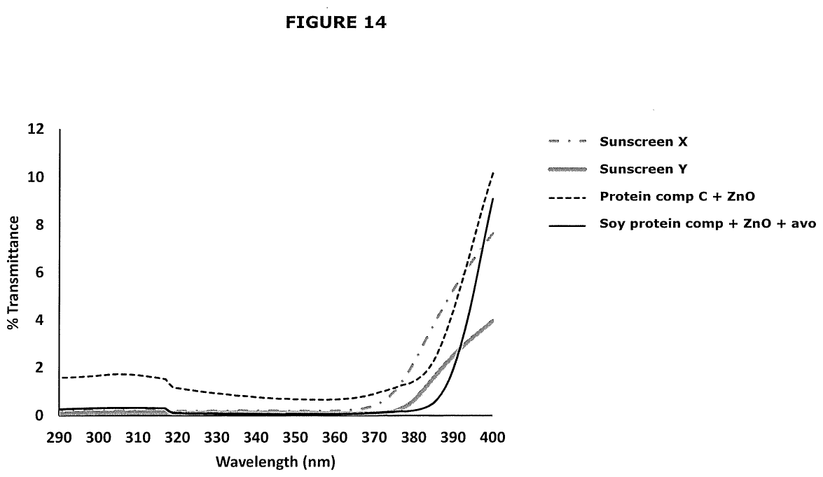

The compositions were applied at a 2 mg/cm2 onto a 42.25 cm2 piece of Vitro-Skin and subjected to 6 wash cycles in water. Each wash cycle consisted of submersion of the skin sample into 650 mL of water, stirred at 200 rpm at 34° C. for 20 minutes. Utilising the UV blocking properties of ZnO (and avobenzone for the soy protein composition), the amount of emulsion remaining on the skin mimetic after washing was determined by measuring the percentage of light transmittance through the skin mimetic (see FIG. 14). In FIG. 14, the dashed grey line labelled “Sunscreen X” represents commercially available sunscreen formulation X with 24.5% zinc oxide; the solid grey line labelled “Sunscreen Y” represents commercially available sunscreen formulation Y with 15% zinc oxide; the dashed black line labelled “Protein comp C+ZnO” represents protein composition C made as per Example 1 with 15% zinc oxide; and the solid black line labelled “Soy protein comp+ZnO+avo” represents the protein composition containing soy protein manufactured using the method described in Example 1 with 15% zinc oxide and with 3% avobenzone. FIG. 14 indicates that both the whey protein composition manufactured as per Example 1 with zinc oxide and the soy protein composition manufactured as per Example 1 with zinc oxide and with avobenzone were suitable at providing coverage of UV radiation across the wavelengths.

The UVA blockage for the following compositions was measured:
commercially available sunscreen formulation X with 24.5% zinc oxide;
commercially available sunscreen formulation Y with 15% zinc oxide;
protein composition C containing whey protein manufactured as described in Example 1 with 10% zinc oxide (ZnO) and 3% avobenzone; and
protein composition C containing soy protein manufactured using the method described in Example 1 with 10% zinc oxide (ZnO) and 3% avobenzone.

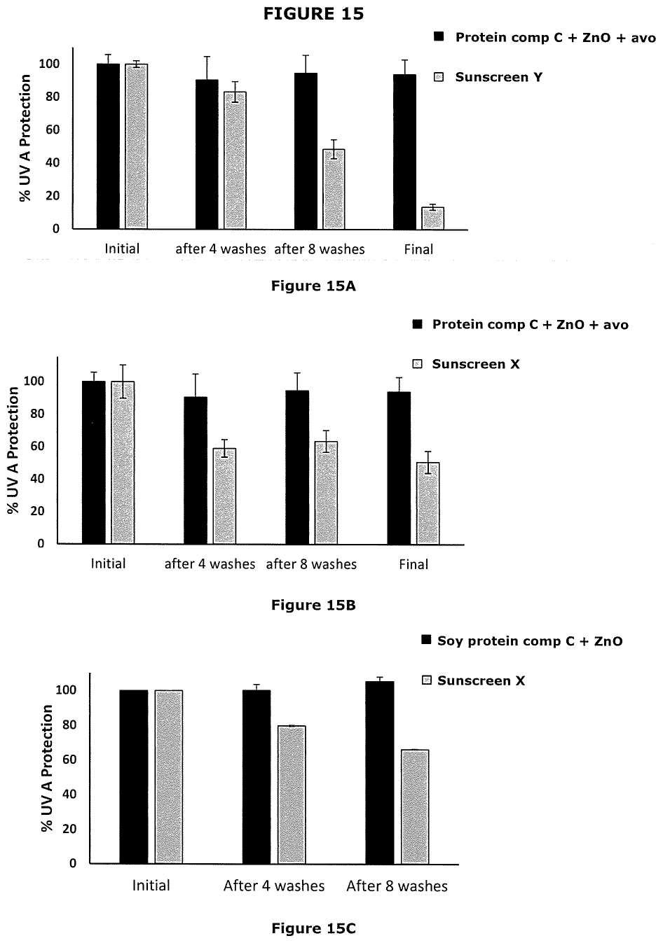

The results are shown in the graphs in FIG. 15 where it can be seen that compositions containing the whey and soy protein compositions made as per Example 1 with zinc oxide and avobenzone provided greater UV blockage (indicated as % UV A Protection) after multiple washing cycles compared to the commercially available sunscreens containing zinc oxide. As the data were normalised to 100% at the start, the results suggest that the protein compositions with ZnO and avobenzone made as described herein were better retained on the skin mimetic compared to the commercially available sunscreens containing ZnO.

### Example 4: Proteolytic Resistance

The proteolytic resistance of the protein compositions produced in Example 1 was measured as follows. Protein compositions produced as described in Example 1 were centrifuged using 100 kD cut off filter, and 50 μL aliquots of retentate were set aside for each experiment. Reaction buffers of Milli Q water with pH adjustment were prepared for the following proteases: for trypsin—pH adjusted to 7.5; for pepsin—pH adjusted to 1.6; and for proteinase K—pH adjusted to 7.5. Stock enzyme solutions for each enzyme (each at 1 mg/mL) were then used to prepare 200 nm solutions of each protease. The proteolytic activity of each protease against the protein composition aliquots was then tested. Analysis by TEM and by Thioflavin T assay (ThT assay) was then performed. The TEM (transmission electron microscope) provides images which can be visually inspected to determine (at least qualitatively) the extent of proteolysis that has occurred. The ThT assay measures changes of influorescence intensity of thioflavin upon binding to amyloid fibrils and thus provides an indication of the extent of proteolysis that has occurred.

Both the TEM and ThT assay results demonstrated that the protein compositions from Example 1 were resistant to degradation by the protease enzymes.

### Example 5: Inhibition of Matrix Metalloproteinase

A colorimetric assay was performed to determine the activity of three matrix metalloproteinases (MMPs): MMP1; MMP2 and MMP9, in the presence of the protein composition C produced as per Example 1.

The experiment was performed as follows. A working stock (1 μM) of each MMP was prepared. A reaction buffer for each MMP was prepared comprising 50 mM HEPES (pH 7.5), 10 mM CaCl2), 150 mM NaCl, and 0.05% Brij 35 (a commercially available nonionic polyoxyethylene surfactant). The MMP (MMP1, MMP2 or MMP9) and buffer were equilibrated for approximately 10 minutes to reaction temperature in a 96-well plate. Samples of the protein composition C (produced as described in Example 1) were added and the plate incubated for 3 hours at 37° C. Table 7 describes the volume of reaction buffer, MMP, protein composition (PC—expressed as the amount of fibrillar material), and fluorogenic substrate used for the controls (negative and positive) and used for each of the test samples.

TABLE 7
Amounts used in test samples and controls

| | Reaction | | | | Total |
|---|---|---|---|---|---|
| | buffer | MMP | PC | Substrate | volume |
| | (μL) | (μL) | (μL) | (μL) | (μL) |
| Blank (negative | 90 | — | — | 10 | 100 |
| control) | | | | | |
| Positive control | 70 | 20 | — | 10 | 100 |
| Test samples | 30 | 20 | 50 | 10 | 100 |

The substrate was then added to each well, and each well read at A_{412 nm} continuously for 30 minutes with 1 minute interval.

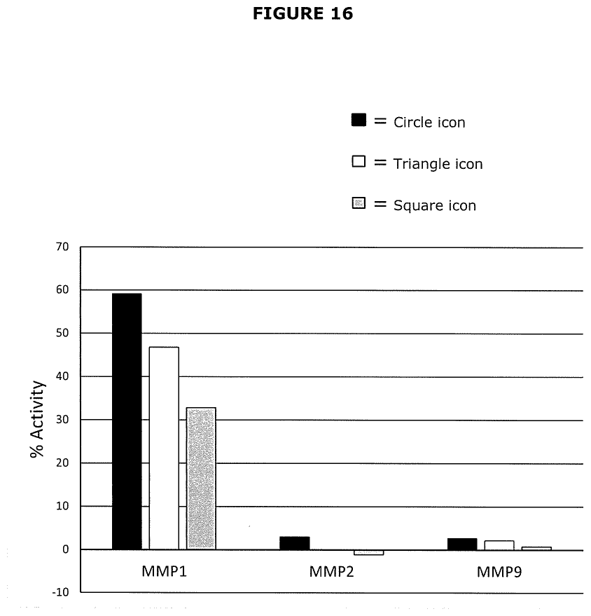

Selected results of this study are shown in FIG. 16, where it can be seen that after 3 hours of incubation, the protein compositions selectively inhibited MMP2 and MMP9 without inhibiting MMP1. In FIG. 16, the % activity=(slope control/slope test sample)×100. In FIG. 16, three separate protein composition C were tested against each of MMP1, MMP2 and MMP9 and are represented by the circle, square and triangle icons, where the circle icon represents a sample of protein composition C that was produced on a small-scale (laboratory bench top) basis, and the square and triangle icons represent samples of protein composition C taken from separate scaled-up batches of protein composition C.

### Example 6: Skincare Products—Facial Night Cream

A skincare product, especially suitable for use as a night cream, was produced with 20% w/w protein composition C (produced as described in Example 1).

The skincare product was produced by making a first mixture of water (˜40% w/w of the final product), sodium hydroxide, vegetable glycerin, and the protein composition C. Separately, a second mixture of ingredients desirable for use in a facial night cream was made, including capric/caprylic triglyceride, sweet almond oil, avocado oil, emulsifiers, thickeners and cetyl alcohol. The two mixtures were (separately) heated to 70-75° C., and the second mixture filtered when decanted into a new vessel. The two mixtures were then combined and homogenised at high shear without aeration, and the pH adjusted if needed. The combined mixtures were then cooled to ≤50° C. and further ingredients added including Manuka honey, water, vitamin E, retinyl palmitate, sodium hyaluronate, vitamin C, fragrance, and a preservative.

The product exhibited levels of bacteria <500 CFU/g, and of fungi <500 CFU/g, and had a pH of 4.5-5.5. The product was also of appropriate texture and consistency for a skin care (especially as a night cream), with a light white colour, and a mild floral fragrance. Rapid absorption of the product into the skin was observed.

### Example 7: Skincare Products—Eye Cream

A skincare product, especially suitable for use as an eye cream, was produced with 20% w/w protein composition C (produced as described in Example 1).

The skincare product was produced by making a first mixture of water (˜47% w/w of the final product), sodium hydroxide, vegetable glycerin, and the protein composition C. Separately, a second mixture of ingredients desirable for use in an eye cream was made, including capric/caprylic triglyceride, sweet almond oil, emulsifiers, thickeners and cetyl alcohol. The two mixtures were (separately) heated to 70-75° C., and the second mixture filtered when decanted into a new vessel. The two mixtures were then combined and homogenised at high shear without aeration, and the pH adjusted if needed (using lactic acid). The combined mixtures were then cooled to 50° C. and further ingredients added including vitamin E, retinyl palmitate, sodium hyaluronate, lactic acid, caffeine, and a preservative.

The product exhibited levels of bacteria <500 CFU/g, and of fungi <500 CFU/g, and had a pH of 5.5-6.5. The product was also of appropriate texture and consistency for a skin care (especially as an eye cream), with a light cream colour, and a mild floral fragrance.

### Example 8: Skincare Products—Day Cream with SPF 15+

A skincare product with SPF 15+, especially suitable for use as a day cream or for specific use for UV protection as a suncream, was produced with 30% w/w protein composition C (produced as described in Example 1).

The skincare product was produced by making a first mixture of water (˜33% w/w of the final product), vegetable glycerin, cationic guar, and the protein composition C. Separately, a second mixture of ingredients desirable for use in a day cream with SPF 15+ was made, including capric/caprylic triglyceride, isopropyl myristate, zinc oxide (at 10% w/w of the final product), butyl methoxydibenzoylmethane, jojoba oil, avocado oil, vitamin C, vitamin E, emulsifiers, thickeners and cetyl alcohol. The two mixtures were (separately) heated to 70-75° C., and the second mixture filtered when decanted into a new vessel. The two mixtures were then combined and homogenised at high shear without aeration, and the pH adjusted if needed (using lactic acid). The combined mixtures were then cooled to 50° C. and further ingredients added including sodium hyaluronate and a preservative.

The product exhibited levels of bacteria <500 CFU/g, and of fungi <500 CFU/g, and had a pH of 6.5-7.5. The product was also of appropriate texture and consistency for a skin care (especially as a day cream with SPF 15+), with a white to off-white cream appearance, and a mild fragrance. Medium absorption of the product into the skin was observed with no whitening effects.

### Example 9: Skincare Products—Mineral Sunblock with SPF 30+

A skincare product with SPF 30+, especially suitable for use as a sunblock, was produced with protein composition C (produced as described in Example 1).

The product contained 50-60% w/w protein composition C, vegetable oil (20-25% w/w), micronized zinc oxide (20-25% w/w), tocopherol (1-3% w/w), cetearyl alcohol (0.5-1% w/w), and fragrance (0.1-0.5% w/w). It was possible to add other ingredients by adjusting the amount of protein composition C and vegetable oil.

It was also found to be a stable and efficacious sunblock, capable of formulation into a water/oil or an oil/water/oil emulsion. The product also exhibited good adherence to the skin to minimise wash-off, and bound UV filters to minimise their absorption into the skin thereby improving UV blocking efficacy. The product exhibited SPF30.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

Although the invention has been described by way of example, it should be appreciated that variations and modifications may be made without departing from the scope of the invention as defined in the claims. Furthermore, where known equivalents exist to specific features, such equivalents are incorporated as if specifically referred in this specification. The specific compositions and methods described herein are representative of preferred examples and are exemplary and not intended as limitations on the scope of the invention. Other aspects and examples will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed as essential. Thus, for example, in each instance described or used herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The assays and methods illustratively described herein suitably may be practiced in differing orders of steps, and that they are not necessarily restricted to the orders of steps indicated herein or in the claims. Further, as used or described herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts disclosed herein may be resorted to by those skilled in the art, and that such modifications and variations are considered to be within the scope of this invention as described herein, and as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Other embodiments are within the following claims. In addition, where features or aspects of the invention are described in terms of Markush groups, those skilled in the art will recognize that the invention is also thereby described in terms of any individual member or subgroup of members of the Markush group.

### REFERENCES

1. Akkermans, C., Van Der Goot, A. J., Venema, P., Gruppen, H., Vereijken, J. M., Van Der Linden, E., and Boom, R. M. (2007). Micrometer-sized fibrillar protein aggregates from soy glycinin and soy protein isolate. Journal of Agricultural and Food Chemistry 55, 9877-9882.
2. Arasteh, A., Habibi-Rezaei, M., Ebrahim-Habibi, A., and Moosavi-Movahedi, A. A. (2012). Response surface methodology for optimizing the bovine serum albumin fibrillation. Protein Journal 31, 457-465.
3. Arnaudov, L. N., and De Vries, R. (2005). Thermally induced fibrillar aggregation of hen egg white Isozyme. Biophysical Journal 88, 515-526.
4. Arnaudov, L. N., De Vries, R., Ippel, H., and Van Mierlo, C. P. M. (2003). Multiple steps during the formation of β-lactoglobulin fibrils. Biomacromolecules 4, 1614-1622.
5. Bhogale, A., Patel, N., Sarpotdar, P., Mariam, J., Dongre, P. M., Miotello, A., & Kothari, D. C. (2013). Systematic investigation on the interaction of bovine serum albumin with ZnO nanoparticles using fluorescence spectroscopy. Colloids and Surfaces B: Biointerfaces, 102, 257-264.
6. Ecroyd, H., Koudelka, T., Thorn, D. C., Williams, D. M., Devlin, G., Hoffmann, P., and Carver, J. A. (2008). Dissociation from the oligomeric state is the rate-limiting step in fibril formation by κ-casein. Journal of Biological Chemistry 283, 9012-9022.
7. Garvey, M., Gras, S. L., Meehan, S., Meade, S. J., Carver, J. A., and Gerrard, J. A. (2009). Protein nano fibres of defined morphology prepared from mixtures of crude crystallins. International Journal of Nanotechnology 6, 258-273.
8. Goda, S., Takano, K., Yutani, K., Yamagata, Y., Nagata, R., Akutsu, H., Maki, S., and Namba, K. (2000). Amyloid protofilament formation of hen egg lysozyme in highly concentrated ethanol solution. Protein Science 9, 369-375.
9. Goers, J., Permyakov, S. E., Permyakov, E. A., Uversky, V. N., and Fink, A. L. (2002). Conformational prerequisites for α-lactalbumin fibrillation. Biochemistry 41, 12546-12551.
10. Gosal, W. S., Clark, A. H., and Ross-Murphy, S. B. (2004). Fibrillar β-lactoglobulin gels: Part 1. fibril formation and structure. Biomacromolecules 5, 2408-2419.
11. Healy, J., Wong, K., Sawyer, E. B., Roux, C., Domigan, L., Gras, S. L., Sunde, M., Larsen, N. G., Gerrard, J., and Vasudevamurthy, M. (2012). Polymorphism and higher order structures of protein nanofibers from crude mixtures of fish lens crystallins: toward useful materials. Biopolymers 97, 595-606.
12. Hettiarachchi, C. A. (2013). β-Lactoglobulin Nanofibrils and Their Interactions with Pectins (Doctoral thesis, The University of Auckland, Auckland, New Zealand). Retrieved from https://researchspace.auckland.ac.nz/handle/2292/21095.
13. Jayawardena N, Kaur M, Nair S, Malmstrom J, Goldstone D, Negron L, Gerrard J A, Domigan L J. (2017 Apr. 11) Amyloid Fibrils from Hemoglobin. Biomolecules; 7(2). pii: E37.
14. Kathiravan, A., Paramaguru, G., & Renganathan, R. (2009). Study on the binding of colloidal zinc oxide nanoparticles with bovine serum albumin. Journal of Molecular Structure, 934(1), 129-137.
15. Kessler J., Yamamoto S., Bour, P. (2017) Establishing the link between fibril formation and Raman optical activity spectra of insulin. Phys Chem Chem Phys (2017) May 31; 19(21): 13614-13621.
16. Konno, T., Murata, K., and Nagayama, K. (1999). Amyloid-like aggregates of a plant protein: a case of a sweet-tasting protein, monellin. FEBS Letters 454, 122-126.
17. Lara, C., Gourdin-Bertin, S., Adamcik, J., Bolisetty, S., and Mezzenga, R. (2012). Self-assembly of ovalbumin into amyloid and non-amyloid fibrils. Biomacromolecules 13, 4213-4221.
18. Loveday, S. M., Wang, X. L., Rao, M. A., Anema, S. G., Singh, H., (2012) β-Lactoglobulin nanofibrils: Effect of temperature on fibril formation kinetics, fibril morphology and the rheological properties of fibril dispersions. Food Hydrocolloids 27, 242-249.
19. Nelson, R., and Eisenberg, D. (2006). Structural models of amyloid-like fibrils. In Advances in Protein Chemistry, Volume 73, Fibrous Proteins: Amyloids, Prions and Beta Proteins (A. Kajava, J. M. Squire and D. A. D. Parry, eds) pp. 235-282. New York: Academic Press.
20. Otte, J., Ipsen, R., Bauer, R., Bjerrum, M. J., and Waninge, R. (2005). Formation of amyloid-like fibrils upon limited proteolysis of bovine α-lactalbumin. International Dairy Journal 15, 219-229.
21. Pearce, F. G., Mackintosh, S. H., and Gerrard, J. A. (2007). Formation of amyloid-like fibrils by ovalbumin and related proteins under conditions relevant to food processing. Journal of Agricultural and Food Chemistry 55, 318-322.
22. Sunde, M., Serpell, L. C., Bartlam, M., Fraser, P. E., Pepys, M. B., and Blake, C. C. F. (1997). Common core structure of amyloid fibrils by synchrotron X-ray diffraction. Journal of Molecular Biology 273, 729-739.
23. Tang, C. H., and Wang, C. S. (2010). Formation and characterization of amyloid-like fibrils from soy β-conglycinin and glycinin. Journal of Agricultural and Food Chemistry 58, 11058-11066.
24. Tang, C. H., Zhang, Y. H., Wen, Q. B., and Huang, Q. (2010). Formation of amyloid fibrils from kidney bean 7S globulin (phaseolin) at pH 2.0. Journal of Agricultural and Food Chemistry 58, 8061-8068.
25. Vetri, V., D'Amico, M., Foderà, V., Leone, M., Ponzoni, A., Sberveglieri, G., and Militello, V. (2011). Bovine serum albumin protofibril-like aggregates formation: solo but not simple mechanism. Archives of Biochemistry and Biophysics 508, 13-24.

1. A skincare composition, a sun care composition or a wound care composition, wherein the composition comprises a proteinaceous component and an agent, wherein the proteinaceous component comprises 5-50 wt % fibrillar protein, and wherein the fibrillar protein comprises a scaffold.

2. The composition of claim 1, wherein the proteinaceous component comprises 10-40 wt % fibrillar protein.

3. The composition of claim 1 or claim 2, wherein the fibrillar protein is derived from whey protein and/or soy protein.

4. The composition of any one of claims 1 to 3, comprising a shear thinning gel.

5. The composition of any one of claims 1 to 4, wherein the agent is selected from the group consisting of vitamin A, vitamin C, vitamin E, hyaluronic acid, sodium hyaluronate, a plant oil, glycerin, salicylic acid, capric/caprylic triglyceride, caffeine, alpha-hydroxy acid, glycolic acid, lactic acid, cetyl alcohol, zinc oxide, avobenzone, an antibiotic, an antimicrobial, a growth factor, a hydrocolloid, and a nonsteroidal anti-inflammatory drug (NSAID).

6. The composition of any one of claims 1 to 5, for use in skincare, sun care, and/or wound care.

7. Use of a composition comprising a proteinaceous component and an agent in the manufacture of a skincare composition, a sun care composition or a wound care composition, wherein the proteinaceous component comprises 5-50 wt % fibrillar protein, and wherein the fibrillar protein comprises a scaffold.

8. Use of a composition comprising a proteinaceous component and an agent in the manufacture of a composition for caring for a keratinaceous surface, for protecting a keratinaceous surface from UV radiation, for protecting a wound, for treating a wound, for preventing an infection in a wound, for treating an infection in a wound, for preventing inflammation in a wound, for reducing inflammation in a wound, for promoting wound healing, and/or for stimulating wound healing, wherein the proteinaceous component comprises 5-50 wt % fibrillar protein, and wherein the fibrillar protein comprises a scaffold.

9. A method for caring for a keratinaceous surface and/or for protecting a keratinaceous surface of a subject from UV radiation, wherein the method comprises administering a composition to the keratinaceous surface of the subject, wherein the composition comprises a proteinaceous component and an agent, wherein the proteinaceous component comprises 5-50 wt % fibrillar protein, and wherein the fibrillar protein comprises a scaffold.

10. A method for protecting a wound, for treating a wound, for preventing an infection in a wound, for treating an infection in a wound, for preventing inflammation in a wound, for reducing inflammation in a wound, for promoting wound healing, and/or for stimulating wound healing in a subject, wherein the method comprises administering a composition to the wound of the subject, wherein the composition comprises a proteinaceous component and an agent, wherein the proteinaceous component comprises 5-50 wt % fibrillar protein, and wherein the fibrillar protein comprises a scaffold.
